(19) United States
(12) Patent Application Publication
(10) Pub. No.: US 2023/0287321 A1
(43) Pub. Date: Sep. 14, 2023

(54) SYSTEMS, METHODS, AND APPARATUS FOR INCREASING BIOREACTOR CAPACITY USING SILICA POLYMERS

(71) Applicant: DRYLET, INC., Houston, TX (US)

(72) Inventors: M. Scott CONLEY, Cypress, TX (US); Mark MENENDEZ, Houston, TX (US)

(21) Appl. No.: 17/966,579

(22) Filed: Oct. 14, 2022

Related U.S. Application Data
(63) Continuation of application No. 15/756,633, filed on Mar. 1, 2018.
(60) Provisional application No. 62/213,094, filed on Sep. 1, 2015.

(51) Int. Cl. C12M 1/12; C02F 3/10; C02F 3/34; C12M 1/00; C12M 1/34; C12N 1/38; C12N 11/14

(52) U.S. Cl. CPC C12M 25/16; C02F 3/107; C02F 3/108; C02F 3/348; C12M 21/12; C12M 41/12; C12M 41/40; C12N 1/38; C12N 11/14

(57) ABSTRACT

Disclosed herein are systems and methods that provide for increased carrying capacity of bioreactors using silica polymers. Disclosed is a method that includes supplying nutrients and silica polymers containing microorganisms to a bioreactor to form a first suspension and controlling temperature, pressure, and nutrient conditions in the bioreactor to produce a second suspension with increased carrying capacity as compared to a control bioreactor containing microorganisms without the silica polymers.

### REFERENCE TO RELATED APPLICATION

This application is a continuation application of U.S. application Ser. No. 15/756,633, filed Mar. 1, 2018, which is the National Stage entry of International Application No. PCT/US2016/050013, filed Sep. 1, 2016, which claims the benefit of U.S. Provisional Application No. 62/213,094, filed Sep. 1, 2015, each of which is herein incorporated by reference in their entireties.

### TECHNICAL FIELD

The present disclosure relates in general to methods, systems, and apparatus for increasing carrying capacity and utilization of bioreactors using silica polymers, specifically systems and methods for increasing organic mass conversion systems using silica polymers. Also disclosed herein are systems and methods for treatment of wastewater using silica polymers.

### BACKGROUND

Bioreactions are utilized in wide range of industrial processes, including but not limited to generation of biofuels, treatment of water, food preparation and processing enterprises such as in making alcoholic beverages, and manufacturing of biological products, such as amino acids and recombinant proteins.

Industrial bioreactions can be operated in a batch mode, a continuous processing mode, or as a hybrid. For example, in the manufacture of therapeutic biologic proteins, batch processing is utilized to obtain stable clinical products at high titers. Continuous bioprocessing is utilized for processes that require, for example, an ongoing evolution of a mixed population of cells that are capable of consuming large amounts of variable feedstock all year around. Continuous bioprocessing is also used in instances where there is production of products that negatively affect cell growth or that are unstable, thus degrade under batch conditions. Water, land, and energy resource management continue to be pressing challenges facing our world, so process optimization of batch, continuous, or hybrid bioprocessing modes is critical to conserving resources and deriving maximum value from current processes utilizing these resources. Process optimization can include increasing operational efficiency, increasing carrying capacity of the bioreaction system, and maximizing yields, while minimizing consumption of raw materials and costs.

As an example, water management involves collection, treatment, and recycling of both clean water and wastewater. Wastewater treatment includes simple accumulation of wastewater followed by discharge of untreated but screened wastewater streams directly to bodies of water, wastewater treatment plants with sophisticated treatment reactors. The products of the treatment processes are primarily clean effluent and solids m the form of sludge. Biological treatment of wastewater is accomplished by growing bacteria m a continuous bioreaction mode under aerobic conditions. Wastewater treatment models focus on global growth rates without regard to the relative abundance of individual species present. In fact, it is impossible to isolate and accurately catalog all of the species present in a wastewater aeration basin. Moreover, the wastewater industry provides the most commonly encountered example of complex mixed culture interactions. For several decades, others have attempted to reduce sludge in wastewater systems by treating with products containing enzymatic blends, liquid based microbial cultures or nutrient based microbial cultures. These have been unsuccessful in reducing sludge. Wastewater sludge consists mostly of water (typically 70-85%). So land application and other potential technologies for energy recovery require that fuel be spent to transport the sludge. Sludge disposal means hauling vast quantities of water around our planet every day. The most preferred waste management practice is not to create the waste in the first place, thus the global objective is to minimize solids production. In this way, sludge reduction represents a movement towards better environmental stewardship and sustainability. In addition, sludge reduction amounts to significant water conservation, as the water content can be returned to the groundwater supply rather than being evaporated thereby contributing to impending water shortages.

Generally, methods involving bioprocessing steps are relatively slow when compared with typical chemical processing steps, as they are typically limited to moderate temperature and pH ranges with relatively dilute streams. Thus, methodologies to accelerate and increase carrying capacity in bioprocessing and microbiologic treatments are needed to increase process efficiency and to better utilize starting resources, effort, and time.

### SUMMARY

Disclosed herein are systems and methods addressing the shortcomings of the art, and may provide any number of additional or alternative advantages. The system and methods described herein provide increased carrying capacity for bioreactors.

Certain embodiments include a method for increasing microbial population. The method includes the steps of supplying nutrients and silica polymers containing microorganisms to a bioreactor to form a first suspension; and controlling temperature, pressure, and nutrient conditions in the bioreactor to produce a second suspension with increased carrying capacity as compared to a control bioreactor containing microorganisms without the silica polymers.

In certain embodiments, the increased carrying capacity of the bioreactor is at least 1.5 times carrying capacity of the control bioreactor containing microorganisms without the silica polymers.

The microorganisms can be aerobic. The microorganisms can be anaerobic. The microorganisms produce a biofuel. The biofuel can be selected from the group consisting of methanol, ethanol, and butanol. In certain embodiments, the silica polymers are precipitated silica granules having a porous structure and loaded with microorganisms throughout the pores of the precipitated silica granules.

Certain embodiments include a method for increasing the carrying capacity of a wastewater treatment facility. The method includes the steps of introducing silica polymers containing microorganisms to a bioreactor containing wastewater to form a first suspension; maintaining the bioreactor under conditions to produce a second suspension, wherein the second suspension has at least two times more total suspended solids than a control bioreactor without application of silica polymers; separating, by a mechanical process, the second suspension to produce a fraction containing suspended solids and a treated water stream, wherein a portion of the fraction containing suspended solids is recycled to the bioreactor.

Another exemplary method includes the steps of introducing wastewater and silica polymers containing microorganisms to a bioreactor to form a first suspension; maintaining the bioreactor under conditions to produce a second suspension, wherein the second suspension has at least two times more total suspended solids than the wastewater stream; separating, by a mechanical process, the second suspension to produce a first fraction containing suspended solids and a treated water stream; separating the first fraction containing suspended solids into a second fraction containing suspended solids and a waste product stream, wherein the second fraction is recycled to the bioreactor. The method can also include the step of adding a flocculating agent to the waste product stream to produce a water stream and a filter cake.

In certain embodiments, the silica polymers containing microorganisms are introduced to a wastewater stream under aerating conditions to form the first suspension. In certain embodiments, the silica polymers are precipitated silica granules having a porous structure and loaded with microorganisms throughout the pores of the precipitated silica granules.

An exemplary method for increasing microbial population includes the steps of supplying silica polymers and nutrients to a bioreactor containing microbes to form a first suspension, wherein the silica polymers provide a substrate for microbial growth; and controlling reaction conditions in the bioreactor to produce a second suspension with increased carrying capacity as compared to a control bioreactor containing microorganisms without the silica polymers.

Certain embodiments include a system for increasing the carrying capacity of a wastewater treatment plant. The system includes an aeration basin configured to mix wastewater and silica polymers containing microorganisms to produce a first suspension; a bioreactor configured to receive the first suspension and produce a second suspension with at least two times more total suspended solids than the wastewater stream; a first solid-liquid separator configured to receive the second suspension from the bioreactor and produce a first fraction containing suspended solids and a treated water stream; and a second solid-liquid separator configured to receive the first fraction containing suspended solids and produce a second fraction containing suspended solids and a waste product stream containing suspended solids, wherein the second fraction is recycled to the bioreactor. In certain embodiments, the silica polymers are precipitated silica granules having a porous structure and loaded with microorganisms throughout the pores of the precipitated silica granules. The system can also include a third solid liquid separator configured to receive the waste product stream and produce a water stream and a filter cake.

Certain embodiments include a system for increasing the carrying capacity of a wastewater treatment plant. The system includes a bioreactor configured to receive silica polymers containing microorganisms and wastewater and produce a suspension with at least two times more total suspended solids than the wastewater; a first solid-liquid separator configured to receive the suspension from the bioreactor and produce a first fraction containing suspended solids and a treated water stream; and a second solid liquid separator configured to receive the first fraction containing suspended solids and produce a second fraction containing suspended solids and a waste product stream containing suspended solids, wherein the second fraction containing suspended solids is recycled back to the bioreactor.

The system can also include a third solid liquid separator configured to receive the waste product and produce a water stream and a filter cake. In certain embodiments, the silica polymers are precipitated silica granules having a porous structure and loaded with microorganisms throughout the pores of the precipitated silica granules.

In certain embodiments, the concentration of mixed liquor suspended solids in the second suspension is greater than 7,000 mg/L. In certain embodiments, the solids retention time of the second suspension in the bioreactor is greater than twenty days.

In certain embodiments, the system for increasing the capacity of a wastewater treatment plant includes a bioreactor configured to receive silica polymers containing microorganisms and a wastewater stream and produce a suspension with at least twice the microbial activity than the wastewater stream; a first solid-liquid separator configured to receive the suspension from the bioreactor and produce a first fraction containing suspended solids and a second fraction containing a treated water stream. The first fraction containing the suspended solids is divided into a third fraction and a fourth fraction, wherein the third fraction is recycled back to the bioreactor and the fourth fraction is forwarded to a second bioreactor for digestion to produce digested products. In certain embodiments, the system further includes a second solid-liquid separator configured to receive the digested products, and remove water from the digested products, and produce a filter cake.

Numerous other aspects, features and benefits of the present disclosure may be made apparent from the following detailed description taken together with the drawing figures. The systems can include less components, more components, or different components depending on desired analysis goals. It should be further understood that both the foregoing general description and the following detailed description are exemplary and explanatory and are intended to provide further explanation of the invention as claimed.

### BRIEF DESCRIPTION OF THE DRAWINGS

The present disclosure can be better understood by referring to the following figures. The emphasis is placed upon illustrating the principles of the disclosure.

FIG. 1 is the average BOD for the Atascocita WWTP facilities exhibited an increasing concentration of BOD (mg/l) over the period from August 2013 through March 2015, according to an embodiment.

FIG. 2 illustrates a trend of decreasing water usage for waste and suggests that water conservation is being achieved through a combination of household utilities and changing water consumption habits, according to an embodiment.

FIG. 3A is the historical trend (higher plotted line) and the test period trend (lower plotted line) that show the number of hauls of sludge per month, according to an embodiment.

FIG. 3B shows the trend for the bio-solids yield and the performance for each month when the WAS rate was increased for a period of time, according to an embodiment.

FIG. 4 is an Ashbook Press existing depreciation line and projected depreciation line.

FIG. 5 is an Andritz Press existing depreciation line and projected depreciation line.

FIG. 6 shows 5-year historical monthly average for power (kWh) use from running the aeration blowers compared against the monthly power consumption (kWh) during the testing period, in accordance with an embodiment.

FIG. 7 shows the 5-year monthly average of total dry solids plotted against the total dry solids produced for each month during the testing period, in accordance with an embodiment.

FIG. 8 illustrates the dramatic and surprising change in microbial growth rates in the aeration basins over an 8 month time period, in accordance with an embodiment.

FIG. 9 illustrates that a new steady state has been achieved, m accordance with an embodiment.

FIG. 10 is a graph of the mass under aeration shows the increasing trend of the total mass during the testing period, in accordance with an embodiment.

FIGS. 11A-D indicate that all of the key performance indicators showed a steady and dramatic increase during the product test period, in accordance with an embodiment.

FIG. 12 shows the analytical results of the effluent during a testing period, in accordance with an embodiment.

FIG. 13 is an image of superior quality sludge, in accordance with an embodiment.

FIG. 14 is a graph of the TSS trend during a test period, in accordance with an embodiment.

FIG. 15 is a batch reactor vessel in accordance with an embodiment.

FIG. 16 is a graphical representation of the growth kinetics of microorganisms growing under certain conditions, in accordance with an embodiment.

FIG. 17 is a graphical representation of the mass to gas percentage increase (about 9% more) under a second set of growth conditions as compared to that under a first set of growth conditions, in accordance with an embodiment.

FIG. 18 is a graphical representation of the rate of sugar consumption under two growth conditions, in accordance with an embodiment.

FIG. 19 is a graphical representation of the logarithmic rate of sugar consumption under two growth conditions, in accordance with an embodiment.

FIG. 20 is a graphical representation of the growth kinetics of microorganisms growing in two conditions, in accordance with an embodiment.

FIG. 21 is a graphical representation of the mass to gas percentage increase under a second set of growth conditions as compared to that under a control set of growth conditions, in accordance with an embodiment.

FIG. 22 is a graphical representation of the rate of sugar consumption under two growth conditions, in accordance with an embodiment.

FIG. 23 is a graphical representation of the logarithmic rate of sugar consumption under two growth conditions, in accordance with an embodiment.

FIG. 24 is a graphical representation of the growth kinetics of microorganisms growing under two growth conditions, in accordance with an embodiment.

FIG. 25 is a graphical representation of the mass to gas percentage increase under a second set of growth conditions as compared to that under a control growth conditions, in accordance with an embodiment.

FIG. 26 is a graphical representation of the rate of sugar consumption under two growth conditions, in accordance with an embodiment.

FIG. 27 is a graphical representation of the logarithmic rate of sugar consumption under two growth conditions, in accordance with an embodiment.

FIG. 28 is a graphical representation of the mannitol production under two growth conditions, in accordance with an embodiment.

FIG. 29 is a graphical representation of the ethanol production under two growth conditions, in accordance with an embodiment.

FIG. 30 is a graphical representation of the sugar uptake under two growth conditions, in accordance with an embodiment.

FIG. 31 is a graphical representation of the growth kinetics of microorganisms growing under two conditions, in accordance with an embodiment.

FIG. 32 is a graphical representation of the mass to gas percentage increase under a second set of growth conditions as compared to that under control growth conditions, in accordance with an embodiment.

FIG. 33 is a graphical representation of the rate of sugar consumption under two growth conditions, in accordance with an embodiment.

FIG. 34 is a graphical representation of the logarithmic rate of sugar uptake under two growth conditions, in accordance with an embodiment.

FIG. 35 is a diagrammatic representation of the experimental set-up designed to study the amount of gasses produced by the anaerobic systems, in accordance with an embodiment.

FIG. 36 is a graphical representation of the average mass rate of gasses produced under the two growth conditions described in Example 2a, in accordance with an embodiment.

FIG. 37 is a graphical representation of the average mass rate of gasses produced under the two growth conditions described in Example 2b, in accordance with an embodiment.

FIG. 38 is a graphical representation of the average mass rate of gasses produced under the two growth conditions described in Example 2c, in accordance with an embodiment.

FIG. 39 is a graphical representation of the average mass rate of gasses produced under the two growth conditions described in Example 2d, in accordance with an embodiment.

FIG. 40 is a graphical representation of the total gas produced per unit vol. of starting culture when there was lower amount food in the system (15 g/l), in accordance with an embodiment.

FIG. 41 is a graphical representation of the rate of gas (cc/hr) generated under three conditions (Reactors 1, 3, and 5) normalized to the amount of unit volume in the starting culture as measured by milliliters (ml), in accordance with an embodiment.

FIG. 42 is a graphical representation of butyric acid production per unit volume of starting culture when there was lower amount food in the system (15 g/l), in accordance with an embodiment.

FIG. 43 is a graphical representation of the rate of butyric acid production per unit volume of starting culture when there was lower amount food in the system (15 g/l), in accordance with an embodiment.

FIG. 44 is a graphical representation of the total gas produced per unit vol. of starting culture when there was lower amount food in the system (15 g/l) but the amount of silica present was increased to 30 g, in accordance with an embodiment.

FIG. 45 is a graphical representation of the rate of gas (cc/hr) generated under three conditions (Reactors 2, 4, and 5 in Table 26) normalized to the amount of unit volume in the starting culture as measured by milliliters (ml), in accordance with an embodiment.

FIG. 46 is a graphical representation of butyric acid production per unit volume of starting culture when there was lower amount food in the system (15 g/l) but the amount of silica present was increased to 30 g, in accordance with an embodiment.

FIG. 47 is a graphical representation of the rate of butyric acid production per unit volume of starting culture when there was lower amount food in the system (15 g/l) but the amount of silica present was increased to 30 g, in accordance with an embodiment.

FIG. 48 is a graphical representation of the total gas produced per unit vol. of starting culture when there was larger amount food in the system (30 g/l), in accordance with an embodiment.

FIG. 49 is a graphical representation of the rate of gas (cc/hr) generated under three conditions (Reactors 6, 8, and 10 in Table 27) normalized to the amount of unit volume in the starting culture as measured by milliliters (ml), in accordance with an embodiment.

FIG. 50 is a graphical representation of butyric acid production per unit volume of starting culture when there was larger amount food in the system (30 g/l), in accordance with an embodiment.

FIG. 51 is a graphical representation of the rate of butyric acid production per unit volume of starting culture, in accordance with an embodiment.

FIG. 52 is a graphical representation of the total gas produced per unit vol. of starting culture when there was larger amount food in the system (30 g/l) and the amount of silica present was increased to 30 g, in accordance with an embodiment.

FIG. 53 is a graphical representation of the rate of gas (cc/hr) generated under three conditions (Reactors 7, 9, and 10 in Table 27) normalized to the amount of unit volume in the starting culture as measured by milliliters (ml), in accordance with an embodiment.

FIG. 54 is a graphical representation of butyric acid production per unit volume of starting culture when there was larger amount food in the system (30 g/l) and the amount of silica present was increased to 30 g, in accordance with an embodiment.

FIG. 55 is a graphical representation of the rate of butyric acid production per unit volume of starting culture, in accordance with an embodiment.

### DETAILED DESCRIPTION

Reference will now be made to the exemplary embodiments illustrated in the drawings, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to one skilled in the relevant art and having possession of this disclosure, are to be considered within the scope of the invention. The present disclosure can be better understood by referring to the attached figures. The components in the figures are not necessarily to scale. The emphasis is instead placed upon illustrating the principles of the disclosure. In the figures, reference numerals designate corresponding parts throughout the different views.

As used here, the following terms may have the following definitions:

“Bioreactor,” as used herein is a system containing microorganisms, in which materials are converted by the microorganisms, or products produced by the microorganisms, or in which increased cell population is achieved. Bioreactors used herein can be one or more of batch reactors, fed-batch reactors, semi-continuous reactors, continuous stirred-tank reactors, continuous flow stirred-tank reactors, and plug-flow reactors, singularly or in series; ebullized-bed (i.e., “bubbling and boiling”) reactors; and fluidized-bed reactors. In certain embodiments, the bioreactor can be an aeration basin. In certain embodiments, the bioreactor can be one or more of a trickling bed reactor, percolating reactors, fluidized reactor, plug-flow reactor, counter-current reactors, sequential batch reactors (“SBR”), and rotating biological contactors.

“Wastewater treatment,” as used herein, refers to a process that converts water that is contaminated water or unsuitable for consumption by plants or animals into an effluent that can be reused for another purpose or returned to the water cycle.

“Silica polymer,” as used herein, refers to precipitated silica granules having a porous structure, super absorbent silica polymers, crystalline silica, fused quartz, fumed silica, silica gels, aerogels, or colloidal silica. In certain embodiments, the silica polymer is precipitated silica granules having a porous structure. For example, suitable precipitated silica granules include those formulated as DryLet® products, such as DryLet® LIFT, DryLet® Aqua Assist, and DryLet® FOG.

“Microorganisms,” as used herein, refers to include bacteria, viruses, mycoplasma, fungi, and protozoa. In certain embodiments, the microorganisms contained in the silica polymers are bacteria. The microorganisms contained in the silica polymers can be a blend of one or more species or genera of bacteria. The microorganism(s) can be selected of one skilled in the art based on the intended use, the available feed sources, and the desired operating conditions for the bioreactor. For example, in a WWTP, these microorganisms do the work of stabilization of organic waste through the production of biomass sludge. Activated sludge treatment relies on native microorganisms present in human flora and in storm water run-off to convert organic material in the influent into new biomass, and old solids or dead biomass in the return activated sludge (“RAS”) into new microorganisms.

“Carrying capacity” of the suspension or a medium, as used herein, refers to the maximum population that a particular bioreaction system can support. In a continuous bioreaction system (a biostat or chemostat), such as a WWTP, it is measured as total suspended solids (“TSS”), mixed liquor suspended solids (“MLSS”), or volatile suspended solids (“VSS”) in units of measure of milligrams per liter. The increased carrying capacity can also be measured by the increased rate of consumption of glucose or other sugars. In a batch system that is not continuously fed with either microbes or food, the carrying capacity can be measured by the peak population density of microbes or alternatively from measuring the rate of growth of the microbes and the rate of consumption of food.

One embodiment of the invention is a composition for delivering microorganisms in a dry mode that contains silica polymers having a porous structure, and microorganisms loaded onto the silica polymer. In another embodiment, microorganisms and the nutrients required for their optimal growth are delivered to a bioreactor with the independent addition of a silica polymer. In another embodiment, the delivery of the microorganisms and nutrients can be achieved by loading the silica polymer with the microorganisms and nutrients to a desired capacity, then applying the loaded product to the bioreactor.

Embodiments of the invention include utilization of the silica polymer loaded with microorganisms in bioreactions occurring in batch reactors. Embodiments of the invention include utilization of the silica polymer loaded with microorganisms in bioreactions occurring in continuous reactors. Embodiments of the invention include utilization of the silica polymer loaded with microorganisms in bioreactions carried out in a hybrid of batch and continuous modes. The bioreactions in the batch, continuous, or hybrid modes can be carried out under aerobic or anaerobic conditions depending on the bioreaction and the organism(s) involved. Embodiments of the invention include utilization of the silica polymer loaded with microorganisms to produce biofuels, including but not limited to methanol, ethanol, or butanol. Embodiments of the invention include utilization of the silica polymer loaded with microorganisms to produce biogas. Embodiments of the invention include utilization of the silica polymer loaded with microorganisms to produce amino acids. Embodiments of the invention include utilization of the silica polymer loaded with microorganisms to produce therapeutically important peptides.

Provided herein are certain embodiments of methods for increasing the carrying capacity of a bioreactor, and also increasing the sustainable utilization of bioreactors using silica polymers. In an exemplary embodiment, the method includes providing a nutrient stream to a bioreactor, and introducing silica polymers containing microorganisms to the bioreactor to form a first suspension. In certain embodiments, the silica polymers containing microorganisms are introduced to the nutrient stream under aerating conditions to form the first suspension before entering the bioreactor. In certain embodiments, the silica polymers are precipitated silica granules having a porous structure and loaded with microorganisms throughout the pores of the precipitated silica granules. The bioreactor containing the first suspension is operated under conditions to form a second suspension that has at least two times more TSS than the first suspension. The second suspension is then subject to a first separation process to produce a first fraction containing suspended solids and the residual liquid stream. The first fraction containing suspended solids is subject to a second separation process to produce a recycle stream back to the bioreactor and a waste product stream containing suspended solids.

An embodiment includes DryLet® LIFT—a proprietary delivery platform for enhanced microbial activity in WWTPs. It is a dry-to-the-touch product that consists of a mixed culture of beneficial microbes immobilized on an inert stratum. For example, the native, non-pathogenic consortium of microbial species is ideal for wastewater application. The microbial species present are not genetically modified strains and belong to the class of Group 1 microorganisms according to the World Health Organization (“WHO”)—microorganisms that are unlikely to cause human disease or animal disease. This product produces blooms of beneficial bacteria when placed into an aqueous environment containing a food source in the form of biomass or dead cells.

A wastewater treatment plant is a continuous process that is modeled as a chemostat. The WWTP must grow as many bacteria as it wastes out, or washout will occur and the WWTP will be emptied of its beneficial bacteria. Growth rates of single species in batch are well understood and are described by Michaelis-Menten kinetics. The bacteria will grow exponentially until the food source is depleted and crowding occurs. Exponential growth is log-linear and corresponds to a very short doubling time for the population. Substrate uptake, or food consumption, is extremely rapid during exponential growth. When the carrying capacity is reached, rapid growth stops and the microorganisms enter a stationary phase. In the stationary phase, the number of bacteria that are “born” equals the number of bacteria that “die” so that the overall population remains unchanged. In this phase, substrate uptake corresponds to a “maintenance” requirement. The population must consume some food just to stay alive. The bacterial population in a WWTP consists primarily of microorganisms in the stationary phase. Eventually, when substrate has been depleted, the population begins to decline by “endogenous decay.” Endogenous decay involves cell lysis and the conversion of dead cell mass into food for other viable bacteria. Endogenous decay depletes the population after all the food is gone. The viable bacteria consume the contents of dead cells in a cannibalistic fashion. Activated sludge treatment capitalizes on this predation by recycling activated sludge back to become food for younger microorganisms in the aeration basin.

Precipitated silica granules are highly porous and contain a huge surface area within their volume and on the surface. For example, the DryLet® LIFT product has approximately about 700,000 square feet per pound in surface area. As with any catalyst, the surface area provides a matrix upon which a reaction can be greatly accelerated. The precipitated silica granules are also a super absorbent polymer that is capable of drawing in organic nutrients to be used as building blocks for new bacterial cells and to sustain cellular functions. As the microorganisms reach exponential growth phase, they experience crowding effects within the silica polymers and begin to populate the surrounding medium.

Provided herein are certain embodiments of methods for increasing the carrying capacity of a WWTP. In an exemplary embodiment, the method includes the steps of introducing wastewater and silica polymers containing microorganisms to a bioreactor to form a first suspension; maintaining the bioreactor under conditions to produce a second suspension, wherein the second suspension has at least two times more total suspended solids than the wastewater stream; separating, by a mechanical process, the second suspension to produce a first fraction containing suspended solids and a treated water stream; separating the first fraction containing suspended solids into a second fraction containing suspended solids and a waste product stream, wherein the second fraction is recycled to the bioreactor. The method can also include the step of adding a flocculating agent to the waste product stream to produce a water stream and a filter cake.

In certain embodiments, the silica polymers containing microorganisms are introduced to a wastewater stream under aerating conditions to form the first suspension. In certain embodiments, the silica polymers are precipitated silica granules having a porous structure and loaded with microorganisms throughout the pores of the precipitated silica granules.

The following key performance indicators are commonly used in the WWTP industry, and have been used here to evaluate the performance of the systems: amount of mixed liquor suspended solids (MLSS), wasted activated sludge (WAS), Volatile Suspended Solids (VSS), Total Suspended Solids (TSS), Recycle Ratio, Return Activated Sludge (RAS), Biological Oxygen Demand (BOD), Dissolved Oxygen (DO), and Sludge Blanket Height. Key performance indicators in WWTP focus on suspended and settled solids. One such indicator is the Solids Retention Time (SRT) because it is undesirable to remove active microorganisms or those in the log phase. If the Solids Retention Time is too long, multicellular organisms or undesired microorganisms become entrenched and affect the system adversely. In cold climates that are not suited for microbial activity, biological treatment is even called “secondary treatment.” In these WWTPs much of the organic waste is never converted to biomass, it is just settled out in primary clarifiers.

In certain embodiments, the yield measured as unit mass of waste product produced per unit mass of organic loading is less than about 40%. In certain embodiments, the yield measured as unit mass of waste product produced per unit mass of organic loading is less than about 30%. In certain embodiments, the yield measured as unit mass of waste product produced per unit mass of organic loading is less than about 20%. Lower yield represents minimization or reduction of biosolids for wastewater treatment.

In certain embodiments, the concentration of MLSS in the second suspension is greater than 7,000 mg/L. In certain embodiments, the concentration of MLSS in the second suspension is greater than 8,000 mg/L. In certain embodiments, the concentration of MLSS in the second suspension is greater than 9,000 mg/L. In certain embodiments, the concentration of MLSS in the second suspension is greater than 10,000 mg/L. In certain embodiments, the concentration of MLSS in the second suspension is greater than 11,000 mg/L. In certain embodiments, the concentration of MLSS in the second suspension is greater than 12,000 mg/L. Increasing MLSS is an important measure for determining the load to a solid liquid separator like a clarifier. Depending on the settling characteristics of the MLSS, which may differ from system to system, it is vital to determine the upper boundary of solids concentration or MLSS being fed to a clarifier or other type of solid liquid separator. The solid liquid separator surface area and the mass rate of suspended solids being introduced into a clarifier allow for the determination of the mass flux, which is a process design parameter for determining the operational size of the clarifier. A higher MLSS also has the added benefit of a higher VSS, which intrinsically has higher microbial activity that benefits the WWTP operations.

In certain embodiments, the solids retention time of the second suspension in the bioreactor is greater than twenty days. In certain embodiments, the solids retention time of the second suspension in the bioreactor is greater than thirty days. In certain embodiments, the solids retention time of the second suspension in the bioreactor is greater than forty days. In certain embodiments, the solids retention time of the second suspension in the bioreactor is greater than forty-five days. In certain embodiments, the solids retention time of the second suspension in the bioreactor is greater than fifty days. In certain embodiments, the solids retention time of the second suspension in the bioreactor is greater than sixty days.

In certain embodiments, the methods described herein include adding a flocculating agent to the waste product stream to produce a water stream and a filter cake. In certain embodiments, the flocculating agent is one or more of an ionic polymer, a non-ionic polymer, and combinations thereof. In certain embodiments, the ionic polymer is a cationic polymer. In certain embodiments, the ionic polymer is an anionic polymer.

In certain embodiments, the reduction of the amount of sludge produced at the WWTP is at least about 40%, when compared to systems that do not employ the silica polymers loaded with microorganisms. In certain embodiments, the reduction of the amount of sludge is at least about 30%. In certain embodiments, the reduction of the amount of sludge is at least about 25%, when compared to systems that do not employ the silica polymers loaded with microorganisms. In certain embodiments, the reduction of the amount of sludge is at least about 20% when compared to systems that do not employ the silica polymers loaded with microorganisms. In certain embodiments, the reduction of the amount of sludge is at least about 15%.

The economic benefit for sludge reduction including savings of financial, time, and personnel resources associated with sludge disposal, consumption of flocculating agents such as polymers, equipment life, and equipment maintenance costs. There are other potential value drivers that may lead to more savings per year and affect the WWTP operational costs, such examples without limitations include lower oxygen demand, higher blower efficiency, decreased qualitative and quantitative use of chemicals for sanitation, and extended WWTP life.

In certain embodiments, the bioreaction system utilizes flocculating agents, which can be one or more of an ionic polymer, a non-ionic polymer, and combinations thereof. Examples include aluminum chloride, ferric chloride and alum. In certain embodiments, the ionic polymer is a cationic polymer, such as agents based on copolymers of AETAC (N,N-Dimethylaminoethyl Acrylate Methyl Chloride Quaternary) or METAC (N,N-Dimethylaminoethyl Methacrylate Methyl Chloride Quaternary) and acrylamide. These products can perform a dual function by both coagulating with their positive ionic charge and flocculating with their high molecular weight. In certain embodiments, the ionic polymer is an anionic polymer, such as agents based on copolymers of acrylamide and acrylic acid, anionic flocculants possess a negative ionic charge and work by binding with residual cationic charges on coagulants adsorbed to coagulated colloids. In certain embodiments, there is at least about 45% reduction in consumption of flocculating agents, when compared to systems that do not employ the silica polymers loaded with microorganisms. In certain embodiments, there is at least about 40% reduction in consumption of flocculating agents, when compared to systems that do not employ the silica polymers loaded with microorganisms. In certain embodiments, there is at least about 35% reduction in consumption of flocculating agents, when compared to systems that do not employ the silica polymers loaded with microorganisms. In certain embodiments, there is at least about 30% reduction in consumption of flocculating agents, when compared to systems that do not employ the silica polymers loaded with microorganisms. In certain embodiments, there is at least about 25% reduction in consumption of flocculating agents, when compared to systems that do not employ the silica polymers loaded with microorganisms. In certain embodiments, there is at least about 20% reduction in consumption of flocculating agents, when compared to systems that do not employ the silica polymers loaded with microorganisms.

Also provided herein are embodiments of systems for increasing the carrying capacity of a WWTP. An exemplary system includes a system for increasing the carrying capacity of a wastewater treatment plant. The system includes an aeration basin configured to mix wastewater and silica polymers containing microorganisms to produce a first suspension; a bioreactor configured to receive the first suspension and produce a second suspension with at least two times more total suspended solids than the wastewater stream; a first solid-liquid separator configured to receive the second suspension from the bioreactor and produce a first fraction containing suspended solids and a treated water stream; and a second solid-liquid separator configured to receive the first fraction containing suspended solids and produce a second fraction containing suspended solids and a waste product stream containing suspended solids, wherein the second fraction is recycled to the bioreactor. In certain embodiments, the silica polymers are precipitated silica granules having a porous structure and loaded with microorganisms throughout the pores of the precipitated silica granules. The system can also include a third solid liquid separator configured to receive the waste product stream and produce a water stream and a filter cake.

Another example includes a bioreactor configured to receive silica polymers containing microorganisms and wastewater and produce a suspension with at least two times more total suspended solids than the wastewater; a first solid-liquid separator configured to receive the suspension from the bioreactor and produce a first fraction containing suspended solids and a treated water stream; and a second solid liquid separator configured to receive the first fraction containing suspended solids and produce a second fraction containing suspended solids and a waste product stream containing suspended solids, wherein the second fraction containing suspended solids is recycled back to the bioreactor. The system can also include a third solid liquid separator configured to receive the waste product and produce a water stream and a filter cake. In certain embodiments, the silica polymers are precipitated silica granules having a porous structure and loaded with microorganisms throughout the pores of the precipitated silica granules. In certain embodiments, the concentration of mixed liquor suspended solids in the second suspension is greater than 7,000 mg/L. In certain embodiments, the solids retention time of the second suspension in the bioreactor is greater than twenty days.

In certain embodiments, the system for increasing the capacity of a wastewater treatment plant includes a bioreactor configured to receive silica polymers containing microorganisms and a wastewater stream and produce a suspension with at least twice the microbial activity than the wastewater stream; a first solid-liquid separator configured to receive the suspension from the bioreactor and produce a first fraction containing suspended solids and a second fraction containing a treated water stream. The first fraction containing the suspended solids is divided into a third fraction and a fourth fraction, wherein the third fraction is recycled back to the bioreactor and the fourth fraction is forwarded to a second bioreactor for digestion to produce digested products. In certain embodiments, the system further includes a second solid-liquid separator configured to receive the digested products, and remove water from the digested products, and produce a filter cake.

Certain key performance indicators from systems that employ various wastewater treatment systems are shown below in Table IA. These values are from a WWTP operator manual, for example provided at http://dca.kv.gov/certification/test%20preparation%20documents/l;vaste‘Vvater’7c.20treatment %20p lant %20operator%20certification %20manual %20-%20revised %20092811.pdf. Also included in Table 1B is a comparison of these performance indicators of an exemplary system employing the silica polymers loaded with the microorganisms.

TABLE 1A
Typical Design Parameters for Activated Sludge Processes

| | | | Organic | | | Return |
|---|---|---|---|---|---|---|
| | | F/M lbs. | Loading | | Detention | Flow to |
| Process | SRT | BOD/lb. | (lbs.BOD/ | MLSS | Time | Plant Flow |
| Modification | (DAYS) | MLVSS/day | 1000 ft³) | (mg/L) | (hours) | Ratio |
| Conventional | 5-15 | 0.2-0.5 | 20-40 | 1000-3000 | 4-8 | 0.25-0.75 |
| Complete Mix | 1-15 | 0.2-1.0 | 50-120 | 1000-6500 | 3-5 | 0.25-1.0 |
| Step Feed | 3-15 | 02.-0.5 | 40-60 | 1500-3500 | 3-5 | 0.25-0.75 |
| Modified Aeration | 0.2-0.5 | 1.5-5.0 | 75-150 | 200-1000 | 1.5-3.0 | 0.05-0.25 |
| Contact | 5-15 | 0.2-0.6 | 60-75 | 1000-3000 | 0.5-1.0 | 0.5-1.5 |
| Stabilization | | | | 4000-9000 | 3-6 | |
| Extended Aeration | 20-30 | 0.05-0.15 | 12.5-15 | 2000-6000 | 18-36 | 0.5-1.5 |
| Oxidation Ditch | 10-30 | 0.05-0.15 | 12.5-15 | 2000-6000 | 18-36 | 0.75-1.5 |
| High Purity | 3-10 | 0.25-1.0 | 100-200 | 3000-8000 | 1-3 | 0.25-0.5 |
| Oxygen | | | | | | |
| Kraus Process | 5-15 | 0.3-0.8 | 40-100 | 2000-3000 | 4-8 | 0.5-1.0 |

TABLE 1B
Typical Design Parameters for Activated Sludge Processes

| | | | | | | Return |
|---|---|---|---|---|---|---|
| | | F/M lbs. | Organic | | Detention | Flow to |
| Process | SRT | BOD/lb. | Loading | MLSS | Time | Plant Flow |
| Modification | (DAYS) | MLVSS/day | (lbs.BOD/1000 ft³) | (mg/L) | (hours) | Ratio |
| DryLet ® | 30-50 | 0.5-0.05 | 20-200 | 7,000-11,000 | 4-17 | 0.5-2.0 |
| process | | | | | | |

Most WWTPs are designed to have a recycle ratio between 50 and 150% of the influent flow rate. The typical range for dissolved oxygen, which is the amount of oxygen that is present in the water, measured in milligrams per liter, and is usually between 2 and 3.5 mg/l in the aeration basin. In most systems in the art, control is achieved by keeping a constant MLSS or a constant solids retention time. The MLSS typically ranges between 2500 and 3500 mg/l. Solids retention time will usually range between 10 and 20 days. The operator will change the wasting rate, which is a fraction of the clarifier underflow to keep a steady-state population, measured as MLSS in the basins. The operator will keep a constant sludge blanket in the clarifiers by changing the RAS or recycle ratio raising the return activated sludge flow rate as blanket height climbs and lowering return activated sludge flow rate if blankets begin to fall.

Most of the VSS into the WWTP (80-90%) are organic foodstuffs like carbohydrates, lipids, and proteins. A small fraction of the VSS coming into the WWTP is composed of nonbiodegradable VSS (“nbVSS”). About 10% of the TSS into the WWTP are composed of inorganic material like metals and silt. Neither the nbVSS nor the inert inorganics will be consumed by biological activity. These solids are not the target of activated sludge treatment. The non-biodegradable solids will simply pass through the WWTP with the vast majority exiting in the generated sludge and a very small amount remaining suspended and exiting at the outfall per limits set by the EPA. Some fraction, fa, of VSS generated in the WWTP remains as non-biodegradable “cell debris.” This cell debris is the major portion of the nbVSS, which along with the inert inorganics comprises sludge and exits the WWTP.

The fraction of total organic carbon (“TOC”) in VSS that is completely biodegradable (1-f_{d}) leaves as carbon dioxide, where fa is the non-biodegradable fraction. The process of waste stabilization involves the oxidation of organic material by bacteria with the production of carbon dioxide and water. Thus, about 50% of the inbound BOD is converted to gas (CO₂ and N₂) and water according to equation below. This is called “burn” or “mass to gas.” Consequently, the biomass synthesis yield is typically less than unity.

$$biomass synthesis yield , Y = \frac{g biomass produced}{g substrate consumed}$$

At WWTP scale, on a day to day basis, one can define the Yield to be the

$$Y = \frac{tons of dry solids out}{tons of BOD in}$$

Yield can vary greatly, but the most efficient WWTPs seem to produce around half a ton of sludge for every ton of biodegradable material they receive. Observed Yield can be much greater in many cases, approaching or even exceeding unity.

Incoming biodegradable material and return activated sludge become food for the microorganisms in the WWTP. The bacteria either use the food for growth (replication) or for cellular maintenance. A small population primarily in stationary phase will use the food to maintain cellular functions (catabolism). A large population primarily in log growth will use the food to produce more cell mass (anabolism). Given a limited food supply, a larger population will undergo more endogenous decay (predation on one another). The decay rate per unit time is increased. In this way, loaded silica polymers drive the system towards greater endogenous decay and cause more mass to leave as gas. And more microbial activity means more highly treated water.

The chemical formula for BOD is C₈H₁₂O₃N₂ Conversion of BOD to cell biomass can be accurately represented by the following balanced chemical equation (Reaction 1).

C₈H₁₂O₃N₂+3O₂→C₅H₇O₂N+3CO₂+H₂O+NH₃

Every 184 grams of BOD treated will produce 113 grams of biomass. This exerts a stoichiometric oxygen demand, which corresponds to three moles of oxygen for every mole of BOD treated. This reaction produces about 0.61 g biomass/g BOD treated. Conversely, 1.42 g of BOD is consumed for every 1 g of biomass produced. Microbial growth produces off gassing of CO₂ and N₂ and generates water.

The complete oxidation of biomass to carbon dioxide, water, and ammonia can be accurately represented by a second balanced chemical equation (Reaction 2).

C₅H₇O₂N+5O₂→5CO₂+2H₂O+NH₃

The first reaction goes essentially to completion, i.e. assume 100% of the inbound BOD is stabilized and converted to biomass during cell growth. However, the second reaction occurs to the extent that consumes the biodegradable fraction (1-fd) of VSS produced in Reaction 1. It is this second reaction that converts VSS mass to gas thereby reducing the observed yield of outbound solids further below the 60% biosolids yield from equation above. If we consider only the first reaction, assume that it goes to completion, and draw a boundary around the aeration basin, then the stoichiometry makes clear some simple overall mass balances around the aeration basins. The mass fraction of TOC in BOD is 96/184 or 52%. In other words, carbon makes up over 50% of the total BOD mass to be treated. Similarly, there is available oxygen contained in BOD. The mass fraction of BOD that is oxygen is 48/184 or 26%.

Carbon does not accumulate, but leaves the WWTP as either gas or sludge. The fraction of TOC in BOD that leaves as gas is 36/96 or 37.5%. The balance of the TOC in BOD that remains captured in biomass is 60/96 or 62.5%. It is this 62.5% that can be further reduced to gas. Of the mass of BOD into the reactor, almost 10% gets reduced to liquid water in the basins. a significant amount of water is generated during microbial growth. Organic nitrogen comprises 28/184 or 15% of the BOD mass load. Half of this 15% gets converted through nitrification/denitrification to N₂ gas in Reaction 1. So adding 37.5% mass to gas due to carbon dioxide and 7.5% mass to gas due to nitrogen, means that about 45% of the total inbound organic load to the WWTP is lost as gas as a result of the first reaction alone. About 45% reduction of BOD mass inbound indicates that the stabilization process (Reaction 1) alone should give a biomass synthesis yield around 55% (or 0.55). Any improvement in the reduction of the yield from the process occurs as the resulting biomass is then further oxidized and gasified through Reaction 2. Reaction 2 describes Endogenous Decay of the biomass generated within the WWTP. Reaction 2, if complete, would convert all the biomass generated into gas and water and half of a mole of ammonia. In this case, sludge would contain only inert inorganics and inert VSS that had entered the WWTP in the influent. Influent streams with greatly different compositional characterization, such as higher loading concentrations of BOD and COD, and streams with much higher inert fraction in the influent may show much higher biomass synthesis yields than the 0.5-0.6 range observed at MUD 109, as described here.

### Example 1

A test facility was selected at the Municipal Utility District (“MUD”) #109 (“MUD109”) in Humble, Tex. This specific WWTP had an excellent record of meeting compliance and had undergone an extensive capital improvement project a few years prior to the study. The MUD109 wastewater treatment facility has an average daily flow rate of 4.1 million gallons. In October 2014, the test began with the introduction of DryLet® LIFT to the headworks of the WWTP at a rate of 8 pounds per day, or a projected annual consumption of 2,920 pounds/year. Samples were collected and analyzed, and flow and operation conditions were monitored for the entire test period. These additional samples and analytics augmented the standard analytics performed by an external laboratory. The test period covered a total of seven (7) months, from October 2014 through April 2015. The test, its methodology, the data analysis, and the results are described herein.

The performance of the DryLet® LIFT product shows an estimated 30% reduction of sludge and a 43% polymer reduction. The economic benefit (see Table 1C) covers four key value drivers for sludge reduction including sludge disposal, polymer use, equipment life, and equipment maintenance costs.

TABLE 1C
Table 1 C - Value Proposition

| | | | | Savings |
|---|---|---|---|---|
| | | | | per Pound |
| | | | | of DryLet ®, |
| | | | | silica polymers |
| | Annual | Annual | Annual | loaded with |
| Value Driver | Cost | Cost | Savings | microorganisms |
| Sludge Disposal | $146,000 | $102,000 | $44,000 | $15 |
| Polymer Use | $35,000 | $19,600 | $15,400 | $5 |
| Equipment Life | $52,000 | $43,000 | $9,000 | $3 |
| Equipment | $35,000 | $23,750 | $11,250 | $4 |
| TOTALS | $268,000 | $188,350 | $79,450 | $27 |

Data from the historical operation of the WWTP was used as the baseline data to compare the data from the system deploying the Dry Let® LIFT product during the test time period. Table 2 presents the various data points collected for the analysis presented herein.

TABLE 2

| | | Aeration | | Outbound |
|---|---|---|---|---|
| | | basin | Effluent | solids |
| Description | Influent data | data | data | data |
| Historical | BOD | MLSS | BOD | Number of |
| From | TSS | SVI | TSS | hauls/month |
| Operator | VSS | Daily | NH₃ | (Assume |
| 2009-2014 | DO | Sample | DO | constant |
| | pH | TSS | pH | 14.27 |
| | 2 samples/ | DO | Daily | wet short |
| | month | BOD | Sample | tons/haul) |
| | Daily Flow | 2 samples/ | | 14.08% solids |
| | Recorded | month | | |
| | everyday | DO | | |
| | | Analyzed | | |
| | | every 3 | | |
| | | minutes | | |
| Test Period | BOD | BOD | BOD | Number of |
| Test | TSS | TSS | TSS | hauls/month |
| America | VSS | VSS | NH3 | (Manifest |
| Inc. | DO | DO | DO | data for |
| Oct 2014- | pH | pH | pH | wet short tons |
| May 2015 | Daily | SVI | Daily | produced |
| | sample | Daily | sample | for each |
| | (M-F) | sample | (as reported | haul) |
| | Daily | (M-F) + | by STS) | 14.08% solids |
| | flow + | STS data | | 38 samples |
| | STS data | (see above) | | over 3 month |
| | (see above) | | | period |
| | | | | collected |
| | | | | by Test |
| | | | | America |

A sludge accounting model focuses on the outbound biosolids production, and relates the mass produced to the mass of BOD load inbound to the WWTP by the Biomass Synthesis Yield calculated herein as Yield=mass of sludge produced per mass of BOD in the influent. The focus of the analysis presented here is predicated on estimates of loads and corresponding solids production, and the goal was to enhance data collection during the test of inbound concentrations and outbound weights. Effluent data remained unchanged and provided proof of ongoing Compliance of Operations. Process data provided a more detailed look at basin dynamics.

Plant Supervisory Control and Data Acquisition have retained the results of two BOD grabs from the Atascocita Joint Operations Board influent for each month dating back 5 years. These samples have always been taken at the beginning and the end of the middle 2 weeks of each month. STS operator's reports also compute an Average Daily Flow rate (ADF) for each month. (Flow rate)×(Concentration) gives mass loading and allows the calculation of an estimated Load to the WWTP during any given month; and, in fact can be averaged over the days of each month to produce an estimated daily load to the WWTP. Given the estimated load to the WWTP during each month of the test and the resulting solids, and those same months' estimated load and solids production from past records, a Yield factor was calculated, which describes higher performance as being associated with a lower yield. Historical biosolids production data for the 5 years preceding the test were limited to the recorded number of hauls per month. These hauls are taken to contain the average tonnage computed during the test period of 14.27 wet tons per haul. For a 3-month period early in the test, the filter cake was analyzed for moisture and percentage solids contents. The cake grabs were only taken on days with no precipitation so that rainfall could not affect the percent moisture. The cake remained almost exactly 85% water content. Solids were reported consistently as 150,000 ppm, or 14% dry solids. As a result, all net wet tonnage of sludge reported on Sludge Haul Manifests was taken to contain:

0.1408×(Net wet sludge in short tons)=(Dry solids in short tons).

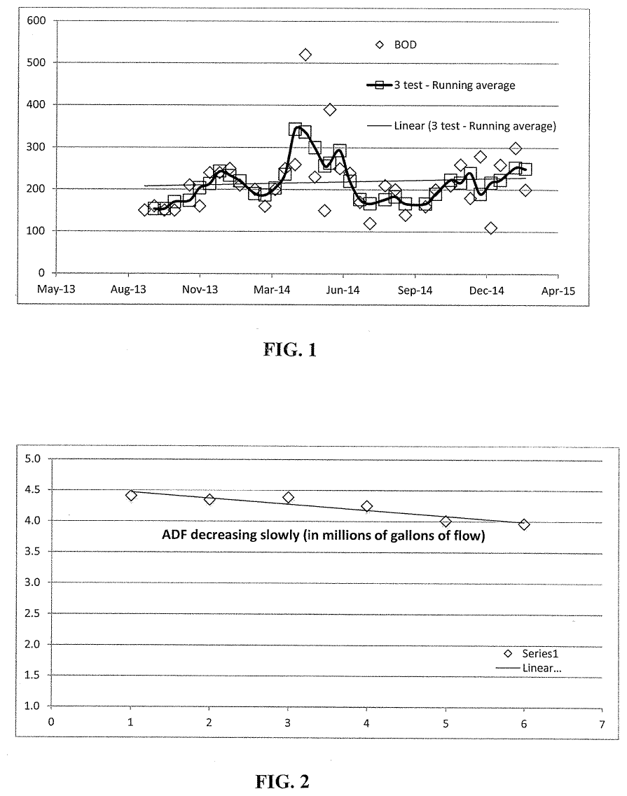

During the test, the influent BOD as measured in milligrams per liter, was recorded far more frequently than in the past using first a composite sampler and later with grab samples. The test sampled for BOD in the influent about 20 times per month, having been scheduled for 5 days a week. As shown in FIG. 1, the average BOD for the Atascocita WWTP facilities exhibited an increasing concentration of BOD (mg/l) over the period from August 2013 through March 2015. However, the average daily flow (ADF) was shown to be decreasing. This suggests higher concentration of BOD related to water conservation utilities in the average home. However, the average total mass of BOD saw little change. The average daily flow (ADF) plot shown in FIG. 2 illustrates a trend of decreasing water usage for waste and suggests that water conservation is being achieved through a combination of household utilities and changing water consumption habits. The mass balances indicate that resulting average BOD load per month has changed very little. The yield analysis takes into account any variability in loading because it calculates a “normalized” load.

The DO in the influent grab samples varies greatly from day to day and may represent cycles of aerobic/anaerobic booms and busts in the collection system pipes. DO grab samples from the basins and the splitter box fell within the operational set point range set by the operators. TSS and VSS in the influent were tracked along with BOD in the influent. (TSS-VSS) gives the Inert Inorganic load to the WWTP. The Inorganic Suspended Solids (TSS-VSS) comprised roughly 10-15% of the solids load. TSS and VSS were also tracked in the basins and at the splitter box. There was no significant change in the inert inorganic fraction to the WWTP or in its basins during the test. Grab samples were extracted from various locations in the WWTP to examine the role of Suspended Solids, BOD, and DO in unit operations, including but not limited to, from the Influent Rapid Mixing Channel, from the aeration basins themselves, and at the splitter box after the basins and before the clarifiers. The grab values reported for the splitter box were used in all basin data tabulation because this is the same location that STS has always used for their bi-monthly basin grabs for TSS and BOD.

The following on-site measurements were recorded-number of bins of sludge filled per day, date for changing a polymer feed drum, “Cook-off” test used to calculate MLSS (mg/l), “Set-test” used to calculate SVI (Sludge volume index) (ml), Blanket height in the 2 clarifiers, and Pounds of product applied each day by STS (8 lbs/day). Finally, Magna Flow records provided manifests describing each 20 yd³ box that was taken for disposal at landfill. Each manifest recorded the Gross Vehicle Weight, Curb Weight, and the Freight on Board, with the waste content appearing as Short Tons. The average weight of a box of sludge was 14.35 tons per 20 yd³ box. Records for the test period and all manifests for previous years produced this same average.

### Yield Analysis

A historical benchmark, or baseline, was established for biosolids yield and then this yield was compared to the yield calculated from WWTP test data with the DryLet® product. Yield calculation is effected by the data sources for Input/Output (I/O) to the mass balance: A simple approach ignores the Input (BOD load) and looks only at the Output (number of hauls) produced during the test (Raw sludge haul accounting).

### Simple Sludge Haul Accounting—Historical Averages and Test Period for Months November Through May

TABLE 3

| | | | Test period | |
|---|---|---|---|---|
| | Historical Average | Test period | (January | Reduced |
| Month | (5 years) | (Actual) | correction) | hauls |
| November | 20 | 15 | 15 | 5 |
| December | 26 | 18 | 18 | 8 |
| January | 22 | 33 | 19* | 3 |
| February | 22 | 15 | 15 | 7 |
| March | 24 | 16 | 16 | 8 |
| April | 28 | 21 | 21 | 7 |

*14 hauls subtracted in January for digester inventory drawdown, 8 to 10 feet of freeboard 2.0% solids

From Table 3, the aggregate number of hauls taken as a historical average for months November through May totaled 142. The total hauls for the same months during the test period totaled 104. The net percent reduction is 26.5% on the number of hauls over the same period from November through May. Excluding January, the net percent reduction is 28.8% over the same period.

### Simple Sludge Haul Accounting—Previous Period (November '13-May '14) and Test Period for Months November Through May

TABLE 4

| | Previous year | | Reduced |
|---|---|---|---|
| Month | (Nov 2013-May 2104) | Test Period | hauls |
| November | 24 | 15 | 9 |
| December | 23 | 18 | 5 |
| January | 26 | 19* | 7 |
| February | 19 | 15 | 4 |
| March | 24 | 16 | 8 |
| April | 46 | 21 | 25 |

From Table 4, the aggregate number of hauls taken as a historical average for months November through May totaled 162. The total hauls for the same months during the test period totaled 104. The net percent reduction is 35.8% on the number of hauls over the same period from November through May. Excluding January, the net percent reduction is 37.5% over the same period.

The following methods were used for yield analysis. A first method uses the same limited data points as the historical record retention to estimate I/O response during the test (Yield analysis). A second method uses a more accurate assessment of the load to the WWTP during the test by sampling BOD more frequently to improve the estimate of I/O response (Yield analysis). A third method uses all the influent BOD sample results to generate a global average of BOD concentration during the test from October to May. This concentration can then be used as the daily concentration for loading calculations throughout the test, and only the ADF would change from day to day (Yield analysis). Another method incorporates actual haul weight data to obtain a more accurate assessment of the Output produced during the test rather than assuming a 14.1 ton average per haul (Yield analysis).

TABLE 5
Table 5 summarizes the differences in the analytic methods
used to calculate Yield of sludge per short ton of BOD:

| Stream | Method 1 | Method 2 | Method 3 |
|---|---|---|---|
| Influent | Operator | Test | Test |
| (Mass In) | analytical: | America BOD | America BOD |
| | Average | analytics: | analytics: |
| | of 2 BOD | monthly | Test period |
| | samples per month | average | (global) average |
| | Daily Flow as | Daily Flow as | Daily Flow as |
| | recorded by | recorded by | recorded by |
| | Operator | Operator | Operator |
| Outbound | Number of hauls | Number of hauls | Number of hauls |
| (Mass Out) | Average weight of | MagnaFlow Inc., | MagnaFlow Inc., |
| | each haul over test | actual manifest | actual manifest |
| | (14.27 short tons per | tonnage | tonnage |
| | haul) | Average percent | Average percent |
| | Average percent | solids | solids |
| | solids | (14.08 | (14.08 |
| | (14.08 | percent dry | percent dry |
| | percent dry | solids per haul) | solids per haul) |
| | solids per haul) | | |

Yield analysis: Historical Average using Method 1. The following tabulated data was calculated using the criterion of Method 1. The percent yield was determined for each month and ranged from 35% to 47%.

$$Monthly Total BOD {( ST )}_{STS , BOD 2 samples} = Σ ( AVG . {BOD}_{2} \times Daily Flow ) \\ Monthly Dry Solids {( ST )}_{AVG , CAKE} = ( Number of Hauls each month ) \times 14.27 [ Average weight of wet sludge per haul in short tons ] \times 0.1408 [ Average mass fraction of solids ]$$

$$Yield ( Method 1 ) = \frac{Monthly Dry Solids {( ST )}_{AVG , CAKE}}{Monthly Total BOD {( ST )}_{STS , BOD 2 samples}}$$

TABLE 6
Historical Average from 2009 through 2014

| | | Average | | Total | Total | Total | | |
|---|---|---|---|---|---|---|---|---|
| | Historical | Accum. | ADF | BOD | Sludge | Dry solids | Percent | # |
| Month | Period | Flow (MGD) | (MGD) | (ST) | (ST) | (ST) | Yield | hauls |
| November | November (′09-′13) | 129 | 4.3 | 100 | 291 | 41 | 41% | 20 |
| December | December (′09-′13) | 138 | 4.4 | 120 | 371 | 52 | 44% | 28 |
| January | January (′10-′14) | 135 | 4.4 | 131 | 316 | 45 | 34% | 22 |
| February | February (′10-′14) | 125 | 4.5 | 96 | 312 | 44 | 46% | 22 |
| March | March (′10-′14) | 136 | 4.4 | 123 | 335 | 47 | 38% | 24 |
| April | April (′10-′14) | 130 | 4.2 | 119 | 395 | 56 | 47% | 28 |
| | Totals | 793 | | 688 | 2020 | 284 | | 142 |
| | | | | | | | w/o January | 119 |

The historical data presented in Table 6 is used to compare the test period data and analysis utilizing Methods 1, 2, and 3. The following sections illustrate the changes in the yield during the test period using DryLet® LIFT.

Yield analysis: Test Period (Method 1) versus Historical Average The following tabulated data was calculated using the criterion of Method 1. The percent yield was determined for each month and ranged from 26% to 37%.

$$Monthly Total BOD {( ST )}_{STS , BOD 2 samples} = Σ ( AVG . {BOD}_{2} \times Daily Flow ) \\ Monthly Dry Solids {( ST )}_{AVG , CAKE} = ( Number of Hauls each month ) \times 14.27 [ Average weight of wet sludge per haul in short tons ] \times 0.1408 [ Average mass fraction of solids ]$$

$$Yield ( Method 1 ) = \frac{Monthly Dry Solids {( ST )}_{AVG , CAKE}}{Monthly Total BOD {( ST )}_{STS , BOD 2 samples}}$$

TABLE 7
Method 1

| | | Average | | Total | Total | Total | | |
|---|---|---|---|---|---|---|---|---|
| | | Accum. | ADF | BOD | Sludge | Dry solids | Percent | # |
| Month | Period | Flow (MGD) | (MGD) | (ST) | (ST) | (ST) | Yield | hauls |
| November | November ′14 | 114 | 3.8 | 114 | 214 | 30 | 26% | 15 |
| December | December ′14 | 126 | 4.1 | 123 | 257 | 36 | 29% | 18 |
| January | January ′15 | 127 | 4.1 | 100 | 266 | 37 | 37% | 19 |
| February | February ′15 | 103 | 3.7 | 110 | 214 | 30 | 27% | 15 |
| March | March ′15 | 130 | 4.2 | 125 | 228 | 32 | 26% | 16 |
| April | April ′15 | 124 | 4.1 | 116 | 300 | 42 | 36% | 21 |
| | Totals | 723 | | 689 | 1479 | 208 | | 104 |
| | | | | | | | w/o January | 85 |

Table 7 presents the comparison of the historical and test period yields using Method 1.

TABLE 8

| | Historical Average | Method 1 | % |
|---|---|---|---|
| Month | Yield | Monthly Yield | Chg. |
| November | 44% | 26% | 40% |
| December | 44% | 29% | 34% |
| January | 35% | 37% | −6% |
| February | 46% | 27% | 40% |
| March | 38% | 26% | 33% |
| April | 47% | 35% | 25% |

As shown in Table 8, the net percent reduction in yields using the averages for each period (historical and test period) results in a 26% decrease. When the yield is calculated on an overall mass balance of BOD load and Dry Solids for the same period the reduction in the yield is 27%. Excluding January 2015 for both cases results in a yield reduction of 33% and 30%, respectively.

Yield analysis: Test Period (Method 2) versus Historical Average. The following tabulated data was calculated using the criterion of Method 2. The percent yield was determined for each month and ranged from 26% to 37%.

$$Monthly Total BOD {( ST )}_{TA , BOD 2 samples} = Σ ( AVG . {BOD}_{20} \times Daily Flow ) \\ Monthly Dry Solids {( ST )}_{ACTUAL , CAKE} = ( Actual Manifest Wet Sludge Weight ) \times 0.1408 [ Average mass fraction of solids ]$$

$$Yield ( Method 2 ) = \frac{Monthly Dry Solids {( ST )}_{ACTUAL , CAKE}}{Monthly Total BOD {( ST )}_{TA , BOD 2 samples}}$$

TABLE 9
Method 2

| | | Average | | Total | Total | Total | | |
|---|---|---|---|---|---|---|---|---|
| | | Accum. | ADF | BOD | Sludge | Dry solids | Percent | # |
| Month | Period | Flow (MGD) | (MGD) | (ST) | (ST) | (ST) | Yield | hauls |
| November | November ′14 | 114 | 3.8 | 109 | 205 | 29 | 26% | 15 |
| December | December ′14 | 126 | 4.1 | 107 | 282 | 40 | 37% | 18 |
| January | January ′15 | 127 | 4.1 | 133 | 266 | 37 | 28% | 19 |
| February | February ′15 | 103 | 3.7 | 115 | 236 | 33 | 29% | 15 |
| March | March ′15 | 130 | 4.2 | 126 | 240 | 34 | 27% | 16 |
| April | April ′15 | 124 | 4.1 | 117 | 302 | 43 | 36% | 21 |
| | Totals | 723 | | 708 | 1530 | 216 | | 104 |
| | | | | | | | w/o January | 85 |

Table 9 presents the comparison of the historical and test period yields using Method 2.

TABLE 10

| | Historical Average | Method 2 | % |
|---|---|---|---|
| Month | Yield | Monthly Yield | Chg. |
| November | 44% | 26% | 40% |
| December | 44% | 37% | 16% |
| January | 35% | 28% | 20% |
| February | 46% | 29% | 37% |
| March | 38% | 27% | 30% |
| April | 47% | 35% | 24% |

As shown in Table 10, the net percent reduction in yields using the averages for each period (historical and test period) results in a 26% decrease. When the yield is calculated on an overall mass balance of BOD load and Dry Solids for the same period the reduction in the yield is 26%. Excluding January 2015 for both cases results in a yield reduction of 28% and 25%, respectively.

Yield analysis: Test Period (Method 3) versus Historical Average. The following tabulated data was calculated using the criterion of Method 3. The percent yield was determined for each month and ranged from 27% to 36%.

TABLE 11
Method 3

| | | Average | | Total | Total | Total | | |
|---|---|---|---|---|---|---|---|---|
| | | Accum. | ADF | BOD | Sludge | Dry solids | Percent | # |
| Month | Period | Flow (MGD) | (MGD) | (ST) | (ST) | (ST) | Yield | hauls |
| November | November ′14 | 114 | 3.8 | 109 | 205 | 29 | 27% | 15 |
| December | December ′14 | 126 | 4.1 | 120 | 282 | 40 | 33% | 18 |
| January | January ′15 | 127 | 4.1 | 121 | 266 | 37 | 31% | 19 |
| February | February ′15 | 103 | 3.7 | 98 | 236 | 33 | 34% | 15 |
| March | March ′15 | 130 | 4.2 | 125 | 240 | 34 | 27% | 16 |
| April | April ′15 | 124 | 4.1 | 118 | 302 | 43 | 36% | 21 |
| | Totals | 723 | | 691 | 1530 | 216 | | 104 |
| | | | | | | | w/o January | 85 |

Table 11 presents the comparison of the historical and test period yields using Method 3.

TABLE 12

| | Historical Average | Method 3 | % |
|---|---|---|---|
| Month | Yield | Monthly Yield | Chg. |
| November | 44% | 27% | 40% |
| December | 44% | 33% | 16% |
| January | 35% | 31% | 20% |
| February | 46% | 34% | 37% |
| March | 38% | 27% | 30% |
| April | 47% | 35% | 24% |

As shown in Table 12, the net percent reduction in yields using the averages for each period (historical and test period) results in a 24% decrease. When the yield is calculated on an overall mass balance of BOD load and Dry Solids for the same period the reduction in the yield is 24%. Excluding January 2015 for both cases results in a yield reduction of 27% and 24%, respectively.

Yield analysis: Steady State Period (Method 3) versus Historical Average. The steady state period is defined by the dates of Mar. 11, 2015 through Apr. 30, 2015. This is the period where the change in TSS in the aeration basin was approximately 9,500±1,500 milligrams per liter or a range of 8,000 to 11,000 mg/L. The following tabulated data was calculated using the criterion of Method 3.

TABLE 13
Yield Analysis for Steady State Period (March, April, & May)

| | Total | Total | Total | | |
|---|---|---|---|---|---|
| | Mass | Mass | Mass | | % |
| | BOD | Sludge | Dry solids | Percent | chg. |
| Data Set | (ST) | (ST) | (ST) | yield | Yield |
| Historical Average | 209 | 641 | 90 | 43% | N/A |
| Steady state test period | 198 | 472 | 67 | 34% | 22.3% |

Table 13 presents the comparison of the historical and test period yields using Method 3.

TABLE 14

| | Historical Average | Method 3 | # |
|---|---|---|---|
| Month | Yield | Monthly Yield | Chg. |
| March-April | 43% | 34% | 22.3% |

As shown in Table 14, the net percent reduction in yields using the averages for each period (historical and test period) results in a 22.3% decrease.

### Operational Factors and Digester Drawdown

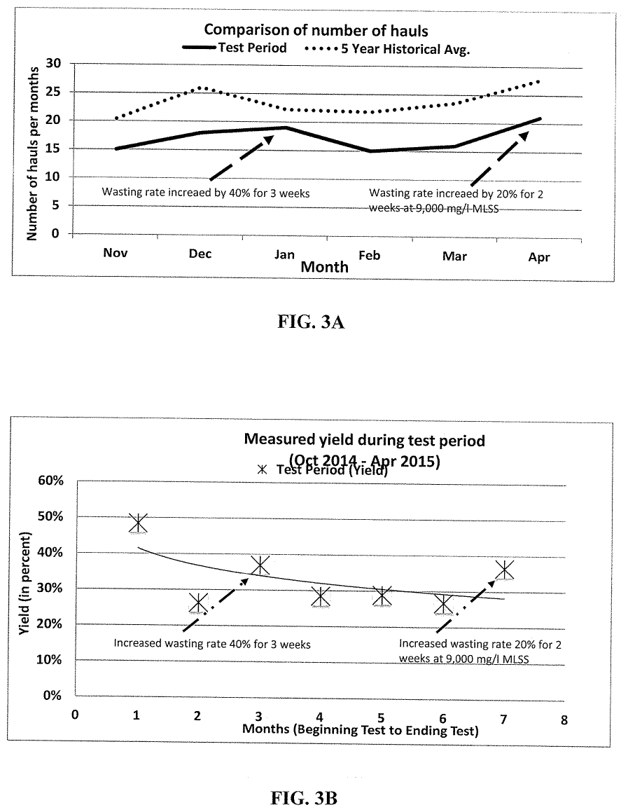

In the last half of December into January, operators decided to draw down their already full digester, as they prefer to operate with some available freeboard capacity as a buffer. In this same period, they raised the wasting rate to 40%. The increased wasting flushed through the system in January and when combined with the digester drawdown, caused a huge spike in hauls in January. The sludge inventory in the digester had to be accounted for. The digester was at full capacity when the study period started and it continued to be full until January. It was then emptied throughout the month as confirmed by the sludge haul manifests. Therefore, the mass of 25 ST of dry sludge that was hauled, but not produced in January was subtracted from the dry metric tons for the month of January. A similar situation occurred for much of April, because there was concern about the high solids levels. These two changes probably resulted in unnecessary sludge production that makes the January and April observed yields both on the high side, as seen in FIGS. 3A and 3B.

The historical trend (higher plotted line) and the test period trend (lower plotted line) in FIG. 3A show the number of hauls per month. The amount of solids wasted is the product of the wasting rate and the wasting concentration. When solids levels are elevated, operators must pay more attention to process control, particularly to the wasting rate. There are two periods where the waste activated sludge (WAS) rate was increased during the period. Interestingly, the plot shows similar seasonality between to the two offset by a total reduction in number of hauls for each and every month. The graph in FIG. 3B shows the trend for the bio-solids yield and the performance for each month when the WAS rate was increased for a period of time. The period of use of DryLet® product reduced the bio-solids yield from a low of 23% to a high of 54%. Month 1 is the comparable month with Month 2 showing the greatest decrease by 54% and month 3 exhibited the least of 23%.

Yield calculated based on the four months was 0.29, resulting in a 32% reduction of sludge. As shown in Table 15, the percent reduction calculated from the various methods gives a range of results from 24% to 37%, with high values corresponding to cases that exclude January, and the lowest values corresponding to the cases in which no raw data was excluded. The results then for sludge reduction can be reported with an extremely high degree of confidence as being 30%+/−8%, and most likely 30%+/−5% with a very good degree of confidence. Average of these calculated values is 28.2%.

TABLE 15

% Reduction

| METHOD | HIGH | LOW |
|---|---|---|
| Sludge haul (Historical) | 28.8 | 26.5 |
| Sludge haul (Last Year) | 37.5 | 35.8 |
| Method 1 | 32.8, 29.8 | 25.8, 26.9 |
| Method 2 | 28.1, 26.4 | 25.1, 26.3 |
| Method 3 | 27.6, 24.6 | 24.4, 24.6 |

### Equipment Life and Maintenance

The reduction of sludge produced has the ancillary effect of extending the life of sludge-handling equipment, namely the belt presses.

### Belt Press Useful Life Extension, Associated Equipment

Replacement of the smaller (Ashbrook) belt press and polymer system is currently estimated at around $667,000 including contingency and engineering. Similarly, the purchase, installation and engineering costs for the larger (Andritz) press totaled approximately $550,000 in 2005 and $672,048 adjusted for inflation.

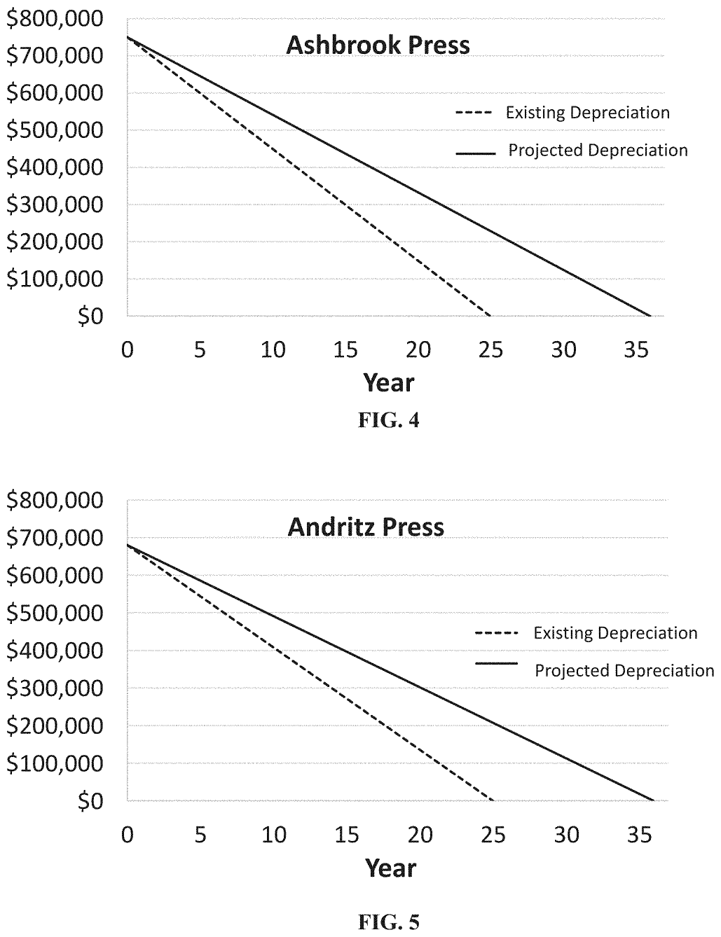

Using the straight-line depreciation method, the capital savings on the presses were estimated to be $8,000 a year for the Ashbrook and $8,070 for the Andritz. The projected depreciation is shown in FIGS. 4 and 5. In FIG. 4, the smaller belt press (Ashbrook Press) depreciation lines are shown for the useful life under standard operating conditions (Existing Depreciation) and the potential useful life under operating conditions while using the product (Projected Depreciation). The reduction in bio-solids through the use of DryLet® product will extend the life of the Ashbrook press by 10 years. In FIG. 5, the larger belt press (Andritz Press) depreciation lines are shown for the useful life under standard operating conditions (Existing Depreciation) and the potential useful life under operating conditions while using the product (Projected Depreciation). The reduction in bio-solids through the use of DryLet® product will extend the life of the Andritz press by 10 years.

To perform the analysis the depreciable asset cost and the straight-line depreciation rate were calculated.

$$Depreciable Asset Cost = Initial Cost - Residual Value \\ Depeciation Rate = \frac{1}{Useful life}$$

They were multiplied to obtain the existing annual depreciation rate:

Existing Annual Depreciation=Depreciation Rate*Depreciable Asset Cost

The annual depreciation rate is then subtracted from the value of the press every year to project its future value. To obtain the projected depreciation the existing depreciation was then multiplied by 70% (reflecting a 30% reduction in use).

Projected Depreciation=Existing Depreciation(1−% reduction in use)

### Belt Press Maintenance Cost Reduction.

The cost savings on belt press maintenance were calculated based on the percentage reduction of biomass yield during the study period. The total amount spent on maintenance and repairs on the belt pressed and associated equipment from the bookkeepers report from fiscal years 2009 to 2013 was adjusted for inflation to 2015 U.S. Dollars and an average yearly cost calculated as shown in Table 16. It was then multiplied by the percent reduction of biomass yield to obtain the 2015 projected expenses. Finally, the 2015 projected savings was calculated by taking the difference of the average and the projected expenses. Assuming a 30% reduction in maintenance and repairs based on the reduction in sludge production from using the DryLet® product, annual savings were thus calculated.

TABLE 16

| | Belt Press | Inflation |
|---|---|---|
| Fiscal year | Maintenance | Adjusted to 2015 |
| June 2009-May 2010 | $21,148 | $23,144 |
| June 2010-May 2011 | $42,175 | $44,744 |
| June 2011-May 2012 | $12,983 | $13,494 |
| June 2012-May 2013 | $66,393 | $68,012 |
| June 2013-May 2014 | $38,412 | $38,721 |
| 5-year Average | $36,222 | $37,623 |
| 2015 Projected Savings (30%) | | $11,287 |
| 2015 Projected Expenses | | $26,336 |

Polymer Used as Flocculating Agent. The polymer, which is used to aid in dewatering the sludge, was supplied just before the presses. While this feed rate is adjustable, it is always entrained into the wasting stream to the presses. A reduction in polymer use occurs naturally from creating less pressed sludge. Historical data for polymer usage was limited to the number of drums purchased within a year prior to the study period. The drums have a volume of 55 gallons, so an average of gallons per month was calculated.

$$Historical Usage ( \frac{gal}{month} ) = \frac{Number of drums invoiced}{month} * 55 ( \frac{gal}{drum} )$$

$$Average drum life ( \frac{day}{drum} ) = \frac{55 ( \frac{gal}{drum} )}{Historical Usage ( \frac{gal}{day} )}$$

Accurate tracking of polymer use was achieved by marking the date the polymer drum was changed. This was performed beginning February 5th and lasting until April 14th. An estimate of gallons per month was then calculated by dividing 55 gallons in a drum over the time period it lasted.

$$Study period usage ( \frac{gal}{drum} ) = \frac{55 gal}{days between drum change}$$

$$Study period usage ( \frac{gal}{month} ) = Study period usage ( \frac{gal}{day} ) * days of the month$$

The study period average was then compared to the average for the previous year to obtain a percent reduction in use.

$$Percent Reduction ( \% ) = \frac{Study period usage ( \frac{gal}{month} ) - historical usage ( \frac{gal}{month} )}{Historical Usage ( \frac{gal}{month} )}$$

The cost savings on polymer usage were calculated based on the percentage reduction of polymer use during the study period. The total amount spent on “Polymer/Sludge Treatment” from the bookkeepers report from fiscal years 2009 to 2013 was adjusted for inflation to 2015 U.S. Dollars and an average yearly cost calculated. It was then multiplied by the percent reduction of polymer use to obtain the 2015 projected expenses. Finally, the 2015 projected savings was calculated by taking the difference of the average and the projected expenses. The polymer, which is used to aid in dewatering the sludge, is fed just before the presses. While this feed rate is adjustable, it is always entrained into the wasting stream to the presses. A reduction in polymer use occurs naturally from creating less pressed sludge. Table 17 shows the historical polymer usage, while Table 18 shows the study period tracking of polymer use.

TABLE 17

| Report Month | Drums | Gallons per Month |
|---|---|---|
| October 2013 | 4 | 220 |
| November 2013 | 4 | 220 |
| December 2013 | 3 | 165 |
| January 2014 | 0 | 0 |
| February 2014 | 3 | 165 |
| March 2014 | 0 | 0 |
| April 2014 | 3 | 165 |
| May 2014 | 5 | 275 |
| June 2014 | 5 | 275 |
| July 2014 | 4 | 220 |
| August 2014 | 0 | 0 |
| September 2014 | 0 | 0 |
| Average | 3 | 142 |

TABLE 18

| Date | Gallons | Gallons per Day | Gallons per Month |
|---|---|---|---|
| Feb. 5, 2015 | N/A | N/A | N/A |
| Feb. 24, 2015 | 55 | 2.89 | 81 |
| Mar. 11, 2015 | 55 | 3.67 | 114 |
| Apr. 14, 2015 | 55 | 1.62 | 50 |
| Average | | 2.73 | 81 |

TABLE 19
Table 19 shows the comparison of the historical and study period
polymer use. A reduction of 43% was documented during
the study period along with a corresponding extension in drum life.

| Polymer Use | Gallons per Month | Drum Life (Days) |
|---|---|---|
| Historical | 142 | 12 |
| Study Period | 82 | 20 |
| % Reduction | −43% | +43% |

TABLE 20
Table 20 shows the cost of Polymer/Sludge Treatment per fiscal year, the
adjustment to 2015 U.S. Dollars and the projected savings for a 43%
reduction in polymer use.

| | Polymer/Sludge | Inflation |
|---|---|---|
| Fiscal year | Treatment | Adjusted to 2015 |
| June 2013-May 2014 | $27,670 | $27,435 |
| June 2012-May 2013 | $71,332 | $71,872 |
| June 2011-May 2012 | $15,613 | $15,962 |
| June 2010-May 2011 | $36,452 | $38,037 |
| June 2009-May 2010 | $19,425 | $20,910 |
| Average | $34,098 | $34,843 |
| 2015 Projected Expenses | | $20,016 |
| 2015 Projected Savings | | $14,827 |

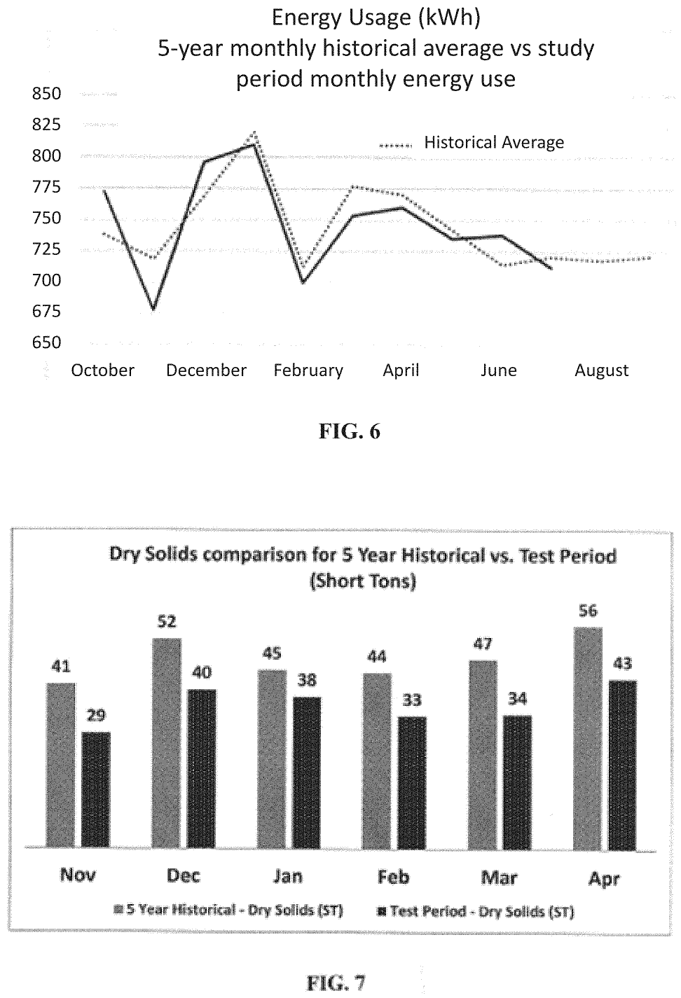

As shown in Table 21, there was minor variation on energy usage on a month-to-month basis and no net change in energy usage over the study period. This can also be seen on FIG. 6, where the 5-year historical monthly average for power (kWh) use from running the aeration blowers was compared against the monthly power consumption (kWh) during the testing period. The overall power use was 2% less than that during the 5-year historical monthly average. This is significant because the use of DryLet® product increased the mass under aeration almost by 300%. One would have expected that more power would have been required significantly through more oxygen consumption. Increased microbial growth increases the blower demand; but in this case, no increase in electrical consumption was detected. Whatever increase in oxygen demand that occurred in the system was offset by operational improvement brought about by blower control tuning.

TABLE 21

| | Historical Average | Study Period | |
|---|---|---|---|
| Month | (2009-2014), in kWh | (2014-2015), in kWh | % Reduction |
| October | 738 | 772 | 5% |
| November | 719 | 678 | −6% |
| December | 768 | 796 | 4% |
| January | 821 | 810 | −1% |
| February | 713 | 700 | −2% |
| March | 777 | 753 | −3% |
| April | 770 | 760 | −1% |
| May | 742 | 736 | −1% |
| June | 715 | 738 | 3% |
| July | 721 | 713 | −1% |
| Average | 748 | 746 | 0% |

### Reduction in Waste Product

In FIG. 7, the 5-year monthly average of total dry solids is plotted against the total dry solids produced for each month during the testing period. For each and every month, the WWTP generated less dry solids for waste disposal. The results of the study show a cumulative and sustained 30% reduction of sludge compared to historical WWTP operations. A variety of methods of analysis all produce the same result indicating a high degree of confidence in the analysis. The study shows that Dry Let® LIFT reduced raw sludge production by 30%+/−5%.

The graph in FIG. 8 illustrates the dramatic and surprising change in microbial growth rates in the aeration basins over the 8 months. The plots represent the TSS and the fraction of the TSS that is the VSS. In the case of the scatter plots, the TSS is the higher values with the VSS just below the corresponding TSS value moving along the time axis. On average, the VSS accounted for approximately 80% of the TSS during the entirety of the test. The use of DryLet® product increased the carrying-capacity of the WWTP almost by 300%. As shown by the plot, the TSS in the aeration basin increased from a normal operating range of 2,500 to 3,500 mg/l to a new range achieved through the use of DryLet® product of 8,000 to 11,000 mg/l. This graph demonstrates the classic sigmoidal shape associated with an increasing microbial population. The population increases from a low level between 0 and 50 to 60 hours and then rapidly increases between about 60 hours to 180 hours, and then plateaus. There are some fluctuations around this pattern, due operational variations, but it follows that trend for both TSS and VSS.

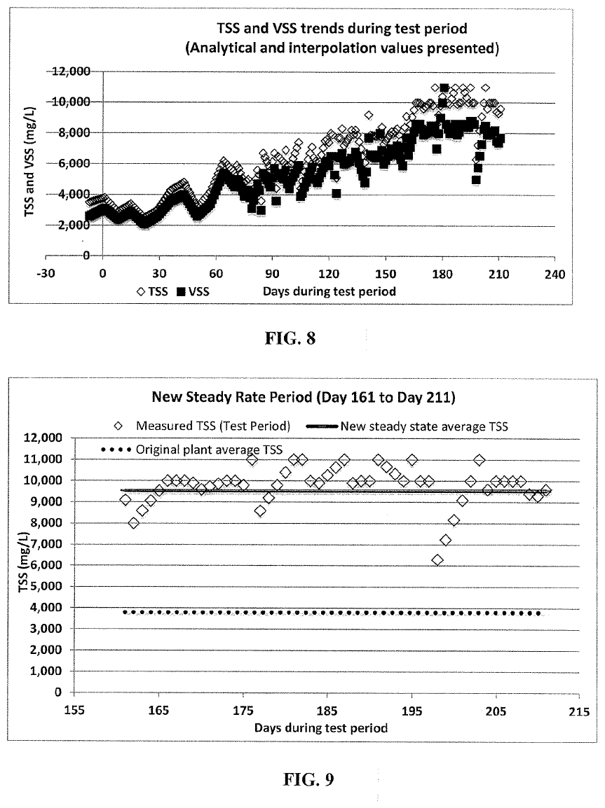

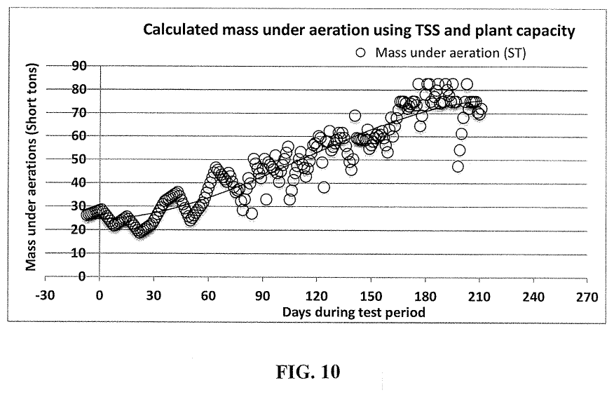

As the microbial population ramps up during a transitional period then mass is accumulated in the system as shown in FIGS. 9 and 10. Focusing on the last three months indicates that a new steady state had been achieved. Original MLSS readings were in the range of 2,500-3,500 mg/l. In the new steady state system, levels were in the range of 9,000-11,000 mg/l for TSS. FIG. 9 shows the significant change in the new operating condition or set point of the WWTP. The lower line shows the average MLSS concentration (mg/l) and the higher line is the average of the scatter plot of data points. Special attention is placed on the large dip in the scatter plot occurring around Day 196, where the WWTP experienced heavy rain events. There was a quick response in the bioreactor as shown by the increase rate of MLSS back to the new set point range of 9,600 mg/1, which was a 2× to 3×increase in WWTP carrying capacity from normal operating conditions. The higher quantity of suspended solids indicates an increase microbial population by a factor of 2.5 from the normal operation with the application of the silica polymer containing microbes. In FIG. 10, the graph of the mass under aeration shows the increasing trend of the total mass during the testing period. The mass under aeration increased by a factor of 3 and sustained this at steady state from about Day 160 to Day 240. There was about a three-fold increase in carrying capacity, which gradually increased from around 20 to 30 tons initially to over 70 to 80 tons in the aeration basin from the start of the test period to about 160 days, when it stabilizes. The increase in carrying capacity was almost three fold.

In this new steady state, over 70 short tons are under aeration in the WWTP. However, after day 160, mass was no longer accumulated in the WWTP. This period shows the same reduction in amount of sludge produced and shows the same reduction in the Yield factor that was observed during the transitional phase. The new steady state was far outside of recommended ranges set forth by regulatory agency guidelines. The WWTP response described here was the result of 8 pounds of DryLet® product a day throughout the test period. In this pilot study, the amount of solids was allowed to climb slowly. It remains to be determined how quickly the carrying capacity can be raised to this level or an even higher level with perhaps a higher dose of the DryLet® product for transitional periods followed by a smaller maintenance dose of 8 pounds.

Either way, the product showed the same 30% reduction after the first month of application. The following savings table was based on the performance of the product after the first month of inoculation projected out to an annual basis. Table 22 focuses on the four main value drivers centered on sludge reduction, and conveys the value proposition for this embodiment of the method and system.

TABLE 22

| Operating Cost/Value Driver | Result |
|---|---|
| Sludge Disposal | 30% Reduction |
| Polymer Usage | 44% Reduction |
| Press Equipment Life | 30% Reduction |
| Press Maintenance | 30% Reduction |

The ancillary benefits of longer equipment life and reduced maintenance on presses and associated equipment naturally arise from simply performing the action of creating 30% less pressed sludge. The polymer, which was used to aid in dewatering the sludge, was fed just before the presses. While this feed rate was adjustable, it was always entrained into the wasting stream to the presses. No extrapolation was required to quantify polymer reduction because operators tracked the rate of consumption. Polymer consumption was reduced 44%.

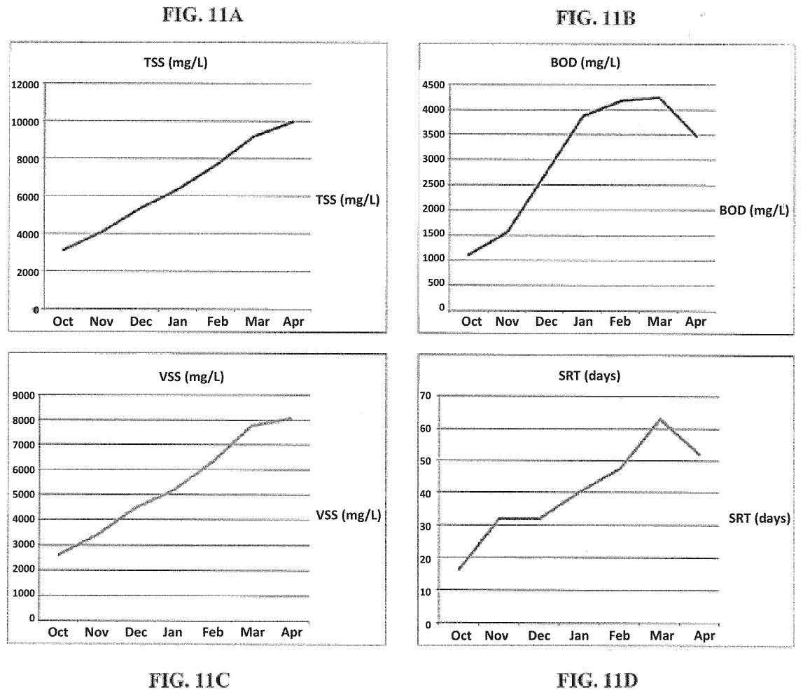

As shown in FIGS. 11A-D, all of the key performance indicators showed a steady and dramatic increase during the product test period. FIG. 11A shows the TSS measured in the aeration basin from October 2014 through April 2015. FIG. 11B shows the BOD measured in the aeration basin from October 2014 through April 2015. FIG. 11C shows the VSS measured in the aeration basin from October 2014 through April 2015. FIG. 11D shows the SRT calculated from the analytical data and wasting rate of the WWTP over the testing period from October 2014 through April 2015.

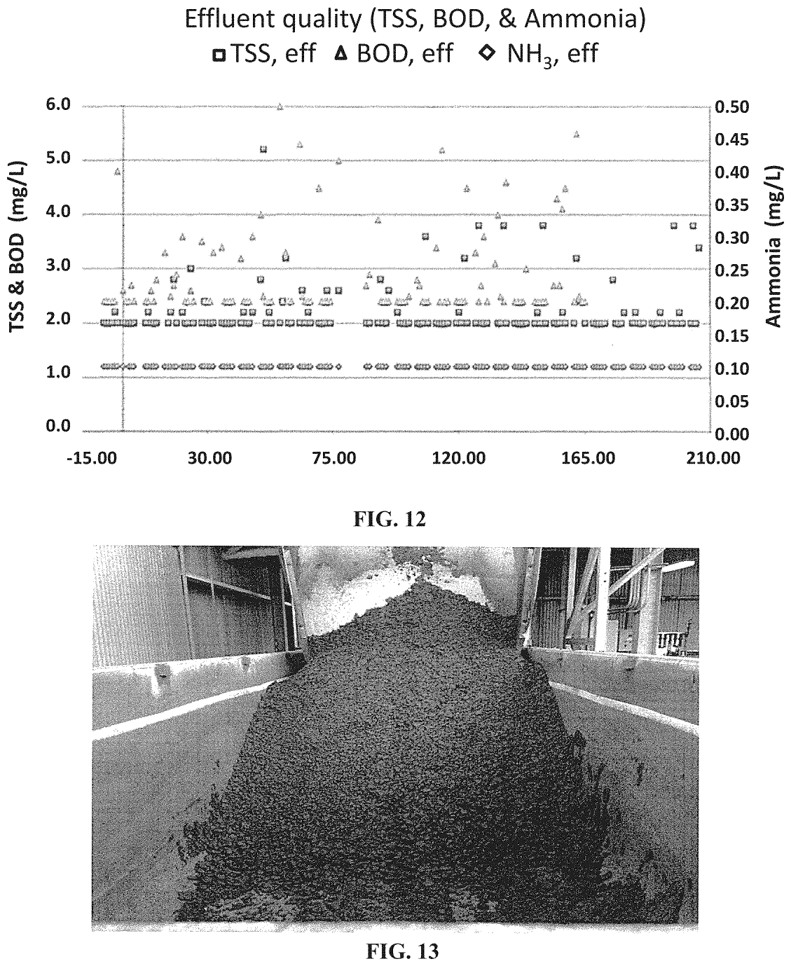

The analytical results of the effluent during the testing period are shown in FIG. 12. The primary y-axis shows the concentration of TSS (represented by squares) and the concentration of BOD (represented by triangles). The secondary y-axis shows the ammonia concentration (represented by triangles). During the testing period the ammonia concentration was below the detection limit of <0.10 mg/1, the TSS concentration was below detection limits for most of the study at a value <2.0 mg/1, and the BOD concentration was the majority of time below the detection limit of <2.5 mg/l. The effluent measurements did not exceed any permitted limit during the testing period. The WWTP remained completely compliant throughout the 7-month test period during all of these changes.

### Effect on Sludge, Polymer, MLSS, BOD, SVI and SRT

There were dramatic changes in several key performance indicators during the course of the product trial with no excursions and no deleterious effects. In fact, the WWTP seemed to run more smoothly with only one full time operator on-site most of the time.

MUD 109 has two presses, a large one and a smaller one. Historically, the “little press” was unable to handle all the solids by itself. However, due to the significantly reduced amount of sludge generated, the smaller press was used almost exclusively during 5 of the 7 months of testing. The larger press required repairs and spent most of the test period off-line. This was apparently not possible before. As a result, the urgency to repair the large press was removed.

Polymer was delivered by an LCM pump into the wasting stream just before the presses. A logbook was maintained on-site to record the number of days that a 55-gallon drum of polymer would last before being emptied and replaced with a new drum. Customarily, one such drum was expected to last about 7-10 days. After the first month of the test, records show that each drum lasted longer; about 14 days on average, and some drums lasted up to 17 days.

Moreover, as shown in FIG. 13, the quality of the sludge was superior. The image shows the sluice of the belt press disposing waste biosolids into a standard waste bin. While the filter cake remained about 85% moisture content, the operational aspects were significantly improved. The cake fell from the belt presses in large sheets of very uniform consistency. Consequently, the operators spent less time hosing the belts down. The man hours saved could be redirected to other operational and maintenance duties.

Using the precipitated silica granules loaded with microorganisms increased several aspects of the WWTP as shown in Table 23. For example, without limitations, sludge production and consumption of flocculating agents reduced 30% and 40% respectively, carrying capacity (MLSS) raised 3.5×, BOD in the basins raised 8-10×, and observed solids retention time increased at least about 5×.

TABLE 23

| Process Variable | Before | | After | |
|---|---|---|---|---|
| TSS in basin | 2,500-3,800 | mg/l | 9,000-11,000 | mg/l |
| DO in basin | 2.0-3.5 | mg/l | 2.0-3.5 | mg/l |
| Recycle Ratio (RAS/Q) | Roughly 100% | | Roughly 100% | |
| Blanket Height | 1 foot in 10 feet SWD | | 1 foot in 10 feet SWD | |
| SRT | 10 | days | 50 | days |
| SVI | Roughly 100 | | Roughly 100 | |
| BOD in basins | 400-500 | mg/l | 3,000-4,000 | mg/l |

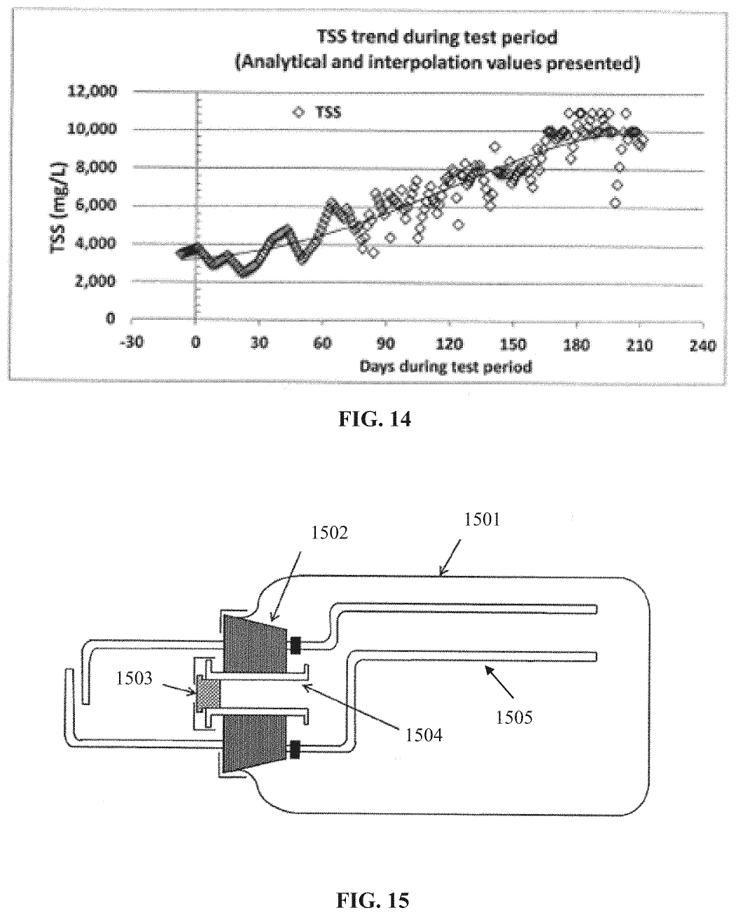

BOD grab samples from the aeration basins also increased dramatically. At the outset, BOD in the basins averaged 400-500. After a few months, this value increased to 3,000 or 4,000 at times. This trend may well support the notion that a much larger microbial population would release far more enzymes and VFAs into the water. These enzymes and VFAs play a significant role in the lysis of inactive biomass, which causes intracellular constituents to become solubilized. The TSS increased as shown in FIG. 14. Lastly, the calculated SRT increased from 9-10 days to a staggering and unexpected 50 days. This value has not been previously known or demonstrated in the industry.

### Effect on Clarifier Solids Flux

While the aeration basins serve as chemostat bioreactors, the secondary clarifiers function strictly for settling suspended solids. Clarifier state point analysis indicated that higher MLSS in the WWTP results in a higher solids loading rate to the clarifiers. Higher mass flux requires that the operator raise the RAS rate to keep a comfortable blanket height. In this WWTP, 2 of 3 clarifiers are in use. Adding the third clarifier would reduce the mass flux through each clarifier 33% over the case with only 2 clarifiers. The entire test period utilized only 2 clarifiers, so it stands to reason that the settling capability of the WWTP was not a limiting factor, even though MLSS more than tripled.

The concern as MLSS increases is that without good settling characteristics, solids would spill out at the overflow. But, in this instance, the clarifiers never failed, and effluent remained in compliance throughout the test. The 30-minute set test was employed on-site as a rough but reliable indicator of good settling characteristics. The 30-minute set was then divided by the MLSS and multiplied by 1,000 to give the SVI. So the solids height after 30 minutes was expected to double if MLSS doubles for the same SVI. But, in this instance, the blanket height also remained constant and presented no challenge to control. STS operators were easily able to adjust RAS and maintain a relatively low blanket height (1 foot) in the presence of a higher solids loading rate, indicating excellent settlability. DO Control and Disinfection

MUD 109 uses a circle chart in the control room to display DO in the aeration basins. Initially, a discrete meter, which took readings only once every 3 minutes, was used at MUD 109 and this had kept the blower use “between the ditches.” The chart began to show a lot of “paint brushing” or cycling on and off frequently as the MLSS increased. This indicated that perhaps the oxygen uptake rate had been significantly increased as the bacterial population became more active. While increased activity suggests a greater oxygen requirement, there was the question of efficiency of oxygen delivery coupled with biological uptake. Oxygen (air) was blown in great excess. But, upon the installation of a continuous meter, the DO charts showed a marked decrease in overshoot and undershoot of the set point range. The frequent cycling stabilized as a result. The new charts with the new continuous meter were much smoother circles showing a far more energy efficient oxygen delivery.

The operators were able to substantially reduce blower requirement by simply staggering set points and shifting both the upper and lower set points down half a point. The tuning capability appeared to relate to accelerated oxygen uptake rate. In fact, having “more horsepower” in a WWTP carries with it the potential to bring about continuous improvement in blower operation and increased process efficiency that would not be possible with a more sluggish microbial population. Ammonia remained below the limit of detection throughout the entire test. BOD was also frequently below MDL. More biological activity corresponds to more highly treated water. This raises the possibility that more microbes and more enzymes in the water might reduce chlorine and dechlor disinfection treatment requirements. These represent an enormous fraction (25%) of the WWTP operating costs. A 10% reduction in chlor/dechlor was very significant. The parameters around sludge and polymer reduction present a clear savings to the WWTP. Furthermore, it is conceivable that blower tuning and chemical disinfection tuning could easily increase the value proposition.

Extending the life of the press by using it 30% less prolongs the equipment's life cycle and delays its inevitable replacement. If municipalities can get more throughputs and more microbial activity with the existing WWTP, the efficiency of the existing wastewater infrastructure is increased. Increasing the effective carrying capacity of an existing WWTP would postpone impending capital improvement projects, particularly those related to expansion and press replacement. The time value of money not spent until later is a positive cash flow over the capital improvement project budgets on financials.

Qualitative Benefits

As stated m the introduction, reducing biosolids makes wastewater treatment more sustainable and more environmentally responsible. Less hauling means less fuel spent and a smaller footprint for disposal. Less hauling also means fewer trucks on the roads and less stress on the infrastructure. The process of pressing the solids is a major function of the operator and consumes a large portion of the time. Helping with ease of operation by reducing the logistics of the outbound solids queue is a logical conclusion. In this way, the product can possibly reduce the total FTE allotted to larger facilities employing several or many workers. A more robust microbial treatment will be more able to absorb BOD step changes and toxic loads. The dose response curve for these events will show a faster dampening factor with increased metabolic activity. While a toxic shock load will still affect some percentage of the population, a larger population will have more viable microorganisms remaining after a kill.

In Example 1, the DryLet® LIFT product displayed a robust impact on WWTP performance in many significant ways. Given the large quantity of data acquired during the test, and the dramatic changes to sludge production and several of the most important process parameters, the beneficial sludge and polymer reduction are attributed to DryLet® LIFT. DryLet® LIFT began to reduce biosolids production after 30 days in the WWTP, and had a sustained 30% reduction of sludge and a 44% reduction of polymer use into the presses. By extension from reduced sludge production, we must associate 2 additional savings to the WWTP from the product: Longer equipment life, and reduced equipment maintenance costs associated with the presses. Deeper examination of bioprocess dynamics reveals Dry Let® LIFT caused an astounding 3-3.5× increase in the carrying capacity of the WWTP in terms of the microbial population as measured by VSS, TSS, and BOD levels in the air-cut water of the basins. The product caused no increase in electrical cost overall, and caused no increase in blower run time. In fact, this study suggests that increased carrying capacity could actually result in blower savings in the future by positively affecting the oxygen uptake rate. The strong positive performance of the DryLet® product for sludge and polymer reduction, combined with the dramatic changes to the process suggest that this technology could have the potential to shift the current understanding of and operation in the Wastewater Industry. This product can reduce the number of basins required at a WWTP because of increased carrying capacity. Methods and products described herein produce reduction in biosolids after about 30 days and realize significant operational and potentially capital improvement project savings while reducing the environmental impact of Wastewater Treatment.

### Additional Supporting Formulae and Modeling Methods

Monthly Sludge Hauls to Short Tons

$$Wet Sludge ( \frac{ST}{month} ) = Sludge Hauls per month * Average Tonnage per Haul$$

Wet sludge to Dry sludge (14% solids)

$$Dry Sludge ( \frac{ST}{month} ) = Wet Sludge * Percentage solids$$

Daily Influent BOD load

$${BOD}_{in} Load ( \frac{ST}{day} ) = {BOD}_{in} ( \frac{mg}{l} ) * ADF ( \frac{M gal}{day} ) * 3.785 ( \frac{Liters}{day} ) * 9.07 * {10}^{8} ( \frac{ST}{mg} )$$

Monthly Influent BOD Load

BODᵢₙ Load(ST/month)=BODᵢₙ Load(ST/day)*days of the month

Monthly Observed biomass yield

$$observed biomass yield , {Y}_{obs} = \frac{g biomass produced}{g substrate consumed} = \frac{Dry Sludge ( ST / month )}{{BOD}_{in} Load ( ST / month )}$$

Yield Percent Reduction

$$Percent Reduction ( \% ) = \frac{{Y}_{obs , hist} - {Y}_{obs , study}}{{Y}_{obs , hist}} = 1 - \frac{{Y}_{obs , study}}{{Y}_{obs , hist}}$$

HRT

$$Hydraulic Retention Time ( day ) = \frac{Volume of Aeration Tank ( {ft}^{3} )}{Influent Flowrate ( \frac{M gal}{day} )} ( \frac{M gal}{{ft}^{3}} )$$

By studying the substrate uptake rate, daily gas production, gas composition and pH, well adapted microbial cultures were established with high predictability with regard to these and other parameters. This also helped in development of a stable balance of buffers, minerals, cofactors and food that would produce manageable amounts of gas given the constraints of high pressure on the experimental reactors. Four different mother cultures of mixed culture microorganisms were maintained over an extended period of weeks. Data was collected over 3 months with good reproducibility observed in the daily observations when the bioreactors were centrifuged, off gassed, sampled, purged and replenished with fresh media, and finally blanketed to be returned to the incubator. These efforts helped design experiments that would be “substrate limited.” In other words, the batches would be limited only by the presence of food, so that a maximum intrinsic growth rate could be established based on the assumption that growth, particularly in early log phase, would be dependent on food concentration and not some other limiting nutrient or toxic byproduct of growth. Also, care was taken to avoid substrate inhibition.

Monod Model describes this saturation kinetics, and works well to model systems with slow growth and a low population density. The Specific Growth rate is First order when the concentration of food is low and then becomes Zero-order at large concentrations of food.

$${μ}_{growth} = \frac{{μ}_{max} s}{{K}_{S} + S} ;$$

where K_{S}=Substrate concentration required to achieve ½ μₘₐₓ.

In any given batch, the initial food concentration is high enough to attribute the observed maximum specific growth rate entirely to the microorganism's intrinsic ability to replicate, or double rapidly in a sustained fashion; just as one can determine a rate of Yield of cell mass from a given amount of a particular substrate for a specific organism (or for a well-trained and highly adapted mixed culture).

Monod Model does not work well to describe very rapid dense populations. Then interactions between species and product inhibition can occur thereby reducing the specific growth rate independent of the effect of substrate.

The Logistic Growth Model is consistent with the Monod Model but takes into account the Lag Phase that always occurs upon dilution of the starter cultures, or inoculums, and is better at predicting the onset of the Declining Phase of a Bloom and ultimately the Carrying Capacity of the batch. The basis of the model is the following differential equation in which the rate of change, or increase in a population, is directly related to the population that is present at that time:

$$\frac{dP}{dt} = rP ( 1 - \frac{P}{K} )$$

By rearrangement, one can see that the Specific Growth rate is linearly related to P (the population present at any given time); and that a plot of

$$( \frac{Δ P}{P} )$$

vs P is linear and monotonically decreasing with a slope of (−r/K) and a y-intercept of r:

$$\frac{Δ P}{P} = r - ( \frac{r}{K} ) P$$

r represents the “boominess” of the system and K is the Carrying Capacity of the system. Together these 2 parameters describe the boom and bust of a batch growth reactor and the maximum final population that can be achieved; which depends on the substrate initially supplied and all other factors influencing growth kinetics, especially the intrinsic growth rate of the species present in the culture. So initial conditions matter immensely in Batch experiments. The above equation can then be integrated to give an analytical expression for the Population as a function of time after r and K have been estimate from the linear fit to the data:

$$P ( t ) = \frac{{KP}_{0}}{{P}_{0} + ( K - {P}_{0} ) {e}^{- rt}}$$

where P₀ is the population at time=0, and r is the growth rate of cells.

This is the logistic equation for transient population increase in a batch reactor. When the data can be fitted well to this model, then population growth is typical of a batch bloom of microorganisms, which display (1) lag phase, (2) exponential (log) phase, (3) steady state or stationary phase, and (4) declining phase.

Here, off-gassing, substrate uptake, and growth associated product formation are studied as direct indicators of microbial growth rates. In the mixed culture experiments, gas composition was analyzed in order to determine the mass of CO₂ and H₂ that were released from each reactor. This release was attributed entirely to either direct or indirect action of the microbes in log growth on the batch reactor. Nitrogen was used to blanket each reactor at every time stamp. Nitrogen production was on parity in all cases. The surprising differences and effects of DryLet® products are evidenced by log growth, which occurred sooner and with much higher log rates of mass to gas than the control situations.

Substrate uptake, when it is limited to only a few sugars, was also used to track microbial growth, and displays predicted logistic behavior. The experiments all focus on capturing data in the early hours which correspond to log growth phases of each culture and each reactor so that we can determine the effect on the observed maximum growth rate when there is no endogenous decay, when secondary blooms have not yet begun, and during which all substrate uptake, gas production, and product formation are directly associated with log growth of the population. The DryLet® product produces a significantly higher intrinsic growth rate as evidenced by the observable parameters.

In any bioprocess, just as in any typical chemical reactor, a higher batch kinetic rate can always be applied to a continuous process to achieve higher throughput, or an increased carrying capacity of a chemostat to produce cell mass more rapidly per unit time. By extension of these observations, the impact of pre-loading and delivery of microorganisms in the form of DryLet® product to any bioprocess chemostat (as in Example 1 at a full scale WWTP) can be optimized to give higher yields of growth associated products, more throughput in terms of the amount of substrate that can be employed without causing substrate inhibition, the more BOD and Chemical Oxygen Demand (COD) that can be consumed per unit time, and an increased carrying capacity of the chemostat at Steady-State.

The Malthusian Model of exponential growth in a batch system (a.k.a. log growth) is given by the simple expression:

N(t)=N₀e^{rt}

in which N represents the number of cells and r is analogous to the same term, which appears in the logistic curve fitting equation. In a chemostat, r (the growth rate of cells) must exceed the dilution rate, or washout occurs. If r>D, then an equilibrium level of cell population, or the carrying capacity will eventually be reached. This will depend on the intrinsic growth rate of the microorganism (r), the Dilution rate (D), the cell mass Yield (Y), and the Half-Concentration commonly denoted by K_{S}.

The “survival equilibrium” in a chemostat, or carrying capacity is given by the following relationship:

$$N = \frac{S - \frac{{K}_{S} D}{r - D}}{Y}$$

In contrast to batch, this survival equilibrium is independent of the starting conditions. The experiments show that one can achieve a lower K_{S} and a higher r. These two parameters in particular account for the increased carrying capacity in Example 1.

Examples 1, 2, and 3 all show higher values for the observed r in several different systems. Lower K_{S} and higher value for r both contribute to an elevated and accelerated population in a chemostat when the DryLet® products are employed. The batch results corroborate the increased carrying capacity that was observed at full scale WWTP.

### Example 2

Batch reactor vessels as shown in FIG. 15 were used to study the carrying capacity of certain embodiments of the systems disclosed herein. Typically, six to nine 1-liter Nalgene centrifuge bottles were used as the fermentation vessels. Each vessel 1501 was fitted with a rubber stopper 1502. A rubber septum 1503 over a glass tube 1504 facilitated the extraction of gas and liquid samples. The vessels have stainless steel rods 1505 that stir the contents as the bottles are rotated in the incubator. Each vessel was charged with roughly 250 ml of total broth, so that each bottle contained 750 ml of headspace to accommodate the large amounts of gas released from each system. The incubator was equipped with rollers that rotate the vessels at 2 rpm. The temperature was held at 40° C. The fermentation vessels were loaded with the same quantity of food source, nutrients, and inoculum. Two sets of three vessels were used for the control and the experiment.

Substrates that are consumed anaerobically produce various organic acids, which can lower the pH of the bioreaction. The pH levels in the reactor were monitored and the acids produced are readily consumed to form acetate and eventually methane and carbon dioxide. The change in pH can be addressed by addition of external acids or bases to maintain optimal conditions for the bioreaction. Gas production volumes and composition of output gases from batch reactors were analyzed, as well as substrate uptake rates and volatile fatty acid production. Samples were extracted during each of the experimental runs, which ranged from 18 to 30 hours in total duration. Gas production, substrate uptake, and VFA production are all correlated with microbial growth. Data was analyzed to determine if preloaded immobilized delivery of an equal inoculum into identical media with equal amounts of food would lead to log growth phase more rapidly and at accelerated rates over control situations.

Two types of growth media were used in the experiments. The first growth medium selected for the methanogenic anaerobic digestion contained only soluble components. The growth medium consisted of a clarified fruit juice (apple juice was used here) that contains nutrients (soluble sugars) and growth factors, such as minerals and vitamins. To maintain near-neutral pH, the juice was supplemented with calcium carbonate and phosphate buffer similar to ATCC Medium 1398 (Modified low phosphate buffered basal medium). The nitrogen source was yeast extract. De-oxygenated water was prepared by boiling the contents pre-loaded with L-cysteine and bisulfite, which also serve as oxygen scavengers. An example media was prepared by adding 200 ml apple juice to 800 ml deoxygenated water. About 5 grams yeast extract powder was added to that and mixed. The pH of solution is then adjusted to 7.5 with the help of 10N sodium hydroxide. Both disodium hydrogen phosphate and potassium dihydrogen phosphate are then added to the media that acts as buffer. The prepared media was then autoclaved and stored.

The second growth medium selected was based on the ATCC formula for Reinforced Methanogens Growth Media. Two mineral solutions—one containing 2.4 g of dibasic potassium phosphate in 400 ml deionized water and another containing 2.4 g each of potassium dihydrogen phosphate and ammonium sulfate, 4.8 g of sodium chloride and 0.6 g of calcium chloride in 400 ml of deionized water—were mixed and diluted to one liter. The pH of the solution was adjusted to 6.35 with the help of 300 μL 10N sodium hydroxide. Wolfe's vitamin solution and Wolfe's mineral solution were added and the pH of the solution was adjusted to 7.35 with 100 μL, 10N NaOH and 1.5 g glycine. The final composition contained about 60 ml of the mineral solution buffer, 8 ml of the yeast solution, 2 ml of 88% formic acid, 5 g of sodium bicarbonate, and 8 ml of each of Wolfe's vitamin solution and Wolfe's mineral solution.

Several distinct mother cultures, prepared and monitored as shown in Table 24, were kept in daily fed batch stasis over a period of 3 months. These mother cultures supplied the inoculums for each experiment, while care was taken to cycle through the stable mother cultures so that no two consecutive experiments depleted the same mother culture.

TABLE 24

| MOTHERS | GBS | | Waco Digester 1 (D 1) | | Waco Digester 2 (D 2) | |
|---|---|---|---|---|---|---|
| Food | 5X dilution fruit juice | | Glucose, sucrose, | | 5X dilution fruit juice | |
| | (~22 g/l) total sugar liquid | | maltodextrin, acid | | (~22 g/l) total sugar | |
| | | | caseinate, ATCC P2 | | liquid | |
| | | | media (~8 g/l total) | | | |
| | | | solids | | | |
| Growth Media | #1 Filtered soluble fruit | | #2 ATCC Growth | | #1 Filtered soluble | |
| | juice with buffer, | | Media for methanogens, | | fruit juice with buffer, | |
| | minerals, growth factors, | | CaCO₃, Drylet ® | | minerals, growth | |
| | CaCO₃ | | silica polymers loaded | | factors, CaCO₃ | |
| | | | with microorganisms, | | | |
| | | | additives* | | | |
| Sampling | Daily (Gas volume, Gas | | Daily (Gas volume, Gas | | Daily (Gas volume, Gas | |
| | GC, weight) | | GC, weight) | | GC, weight) | |
| Daily Purge | 100 | ml | 50 | ml | 80 | ml |
| Amount | | | | | | |
| Avg. cc/day | 900-1100 | cc/day | 1300-1700 | cc/day | 1000-1200 | cc/day |
| Avg. cc/hr | ~40 | cc/hr | ~60 | cc/hr | ~45 | cc/hr |

| | | | |
|---|---|---|---|
| Avg. CO2% | 50-30% | 15-30% | 15-30% |
| Avg. N2% | 60-80% | 60-80% | 60-80% |
| Avg. CH4 | 0-10% | 0-10% | 0-10% |
| Avg. H2% | 5-20% | 5-20% | 5-20% |
| Avg. pH | 4.25-7.5 | 5.25-7.5 | 5.75-7.5 |

Note:
pH was adjusted each day to 7.5.

The inoculum for each reactor was a 5 ml aliquot of a homogenous broth, extracted via pipette after mixing the vessel of the mother culture well; and each aliquot was supplied to one of each of the reactors. In this way, each reactor received a nearly an identical number of viable cells that were all exposed instantaneously to a 50×dilution with regard to cell concentration. This “shock” always resulted in a lag phase for growth, which ranged from 4-6 hours.

The volume of gas produced in the vessels was measured by displacing an aqueous solution of CaCl₂) in a graduated water column. In this instance, gas volumetric measurement was carried out by calculating the volume of gas (cc) produced at each time stamp along with the 750 ml headspace in each reactor and the 30 cc sample removed for the gas chromatography. The composition of the gas (methane, carbon dioxide, hydrogen, nitrogen) was measured by gas chromatography. Sugar concentration from centrifuged 2 ml samples was analyzed using a High Pressure Liquid Chromatography (HPLC) system and UV detector. Gas-liquid chromatography was used to measure acid production by concentration from centrifuged 2 ml samples.

Three replicates for each of the two conditions—control and treatment with silica microspheres loaded with microorganisms—constituted six bottles under identical experimental conditions and identical sampling regimens. Each of these six bottles was filled with the same 250 mL of growth medium. In each case, the total amount of broth used to inoculate each bottle was 5 mL; however, under conditions using the precipitated silica containing microorganisms, 5 ml of the culture broth was mixed with an equal fraction of media, and then the resulting solution was absorbed onto silica prior to being charged into the reactor.

### Example 2a

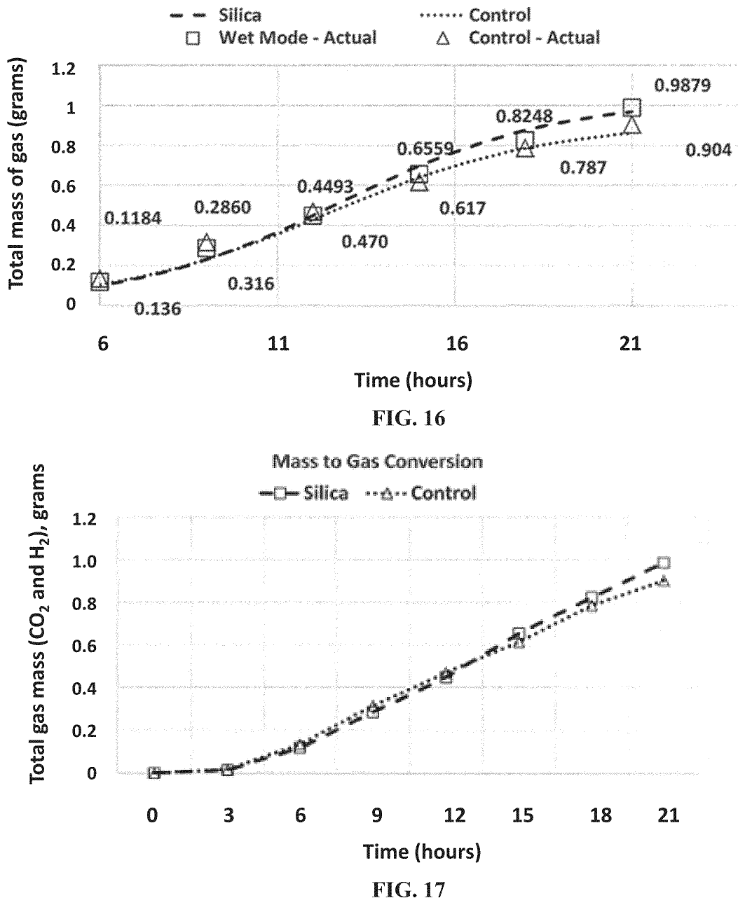

The growth kinetics of microorganisms growing under two conditions were examined first, microorganisms that have been introduced to the bioreactor as an inoculum from the mother culture; and second, microorganisms that have been introduced to the bioreactor as an inoculum from the mother culture along with the addition of five grams of silica polymer to the bioreactor. The energy solution consisted primarily of about six grams of glucose, sucrose, and fructose. FIG. 16 is a graphical representation of the growth kinetics of microorganisms growing under these conditions. Both cultures entered log phase in about six hours. These sigmoidal plots of population growth show the similarities in growth profiles of the microorganisms under the two conditions.

In these experiments, the anaerobic bacteria converted the nutrients m the growth medium into volatile acids and then into biogas-a gas composed of methane and carbon dioxide, and trace amounts of water vapor, hydrogen sulfide, and ammonia. FIG. 17 is a graphical representation of the mass to gas percentage increase (about 9% more) under the second growth conditions as compared to that under the first growth conditions.

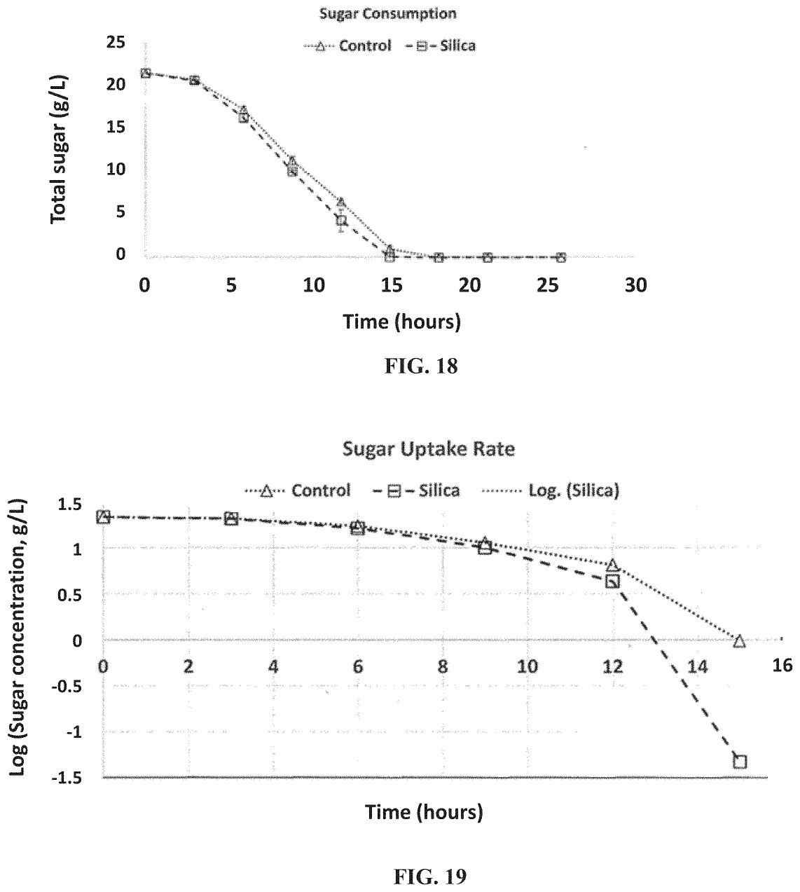

FIG. 18 is a graphical representation of the rate of sugar consumption under the two growth conditions. There was a slightly increased rate of consumption of sugars by bacteria growing in the presence of the silica polymers added to the bioreactors. While the sugar uptake reached completion around 18 hours under both conditions, about 60% of the sugars were consumed by the microorganisms in 9 hours growing in the presence of the silica polymers added to the bioreactors versus the same 60% of the sugars were consumed by the microorganisms in 11.5 hours growing under the control conditions.

FIG. 19 is a graphical representation of the logarithmic rate of sugar consumption under the two growth conditions. By 15 hours, nearly all of the sugar had been consumed in both reactors. The rate of sugar consumption in the control reactor of 1.40 g/l/hr and the microbes growing in the presence of the silica polymers had a rate of sugar consumption of 1.46 g/l/hr. The control underperformed by 4.3% relative to the silica reactor.

### Example 2b

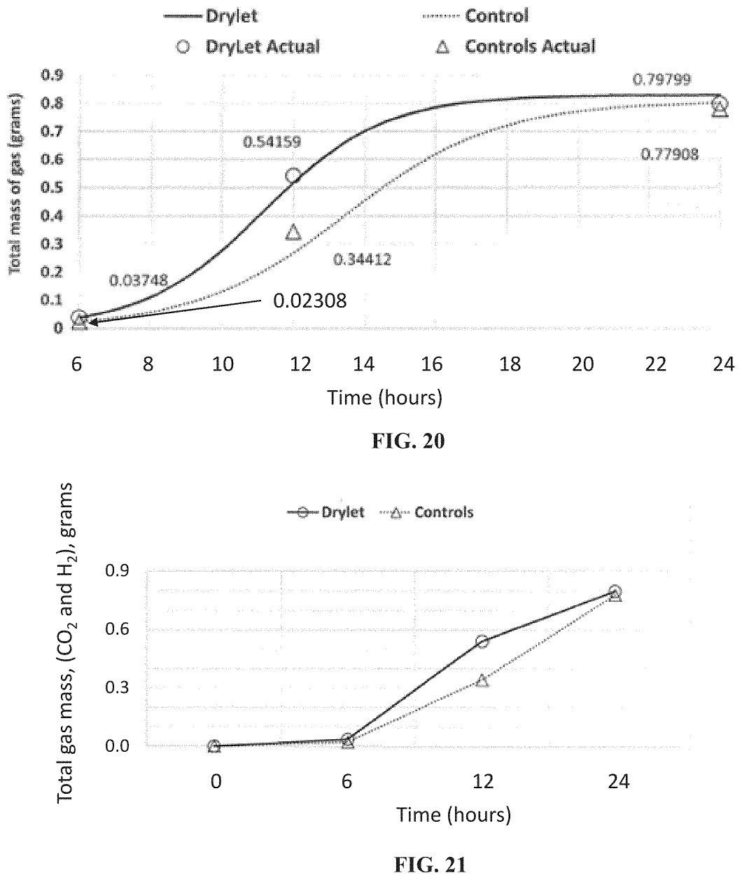

The growth kinetics of microorganisms growing under another set of two conditions were examined-first, microorganisms that have been introduced to the bioreactor as an inoculum from the mother culture; and second, same amount of microorganisms that have been loaded onto 6.5 grams of precipitated silica granules and then introduced to the bioreactor. Compared to the previous sets of growth conditions, the nutrient solution consisted primarily of about four grams of glucose, sucrose, maltodextrin, and P2 media. FIG. 20 is a graphical representation of the growth kinetics of microorganisms growing these two conditions. The microorganisms loaded onto precipitated silica granules entered log phase in about five hours, while the microorganisms under the control conditions entered log phase about two hours later.

FIG. 21 is a graphical representation of the mass to gas percentage increase under the second growth conditions as compared to that under the control growth conditions. There was a 54% increase in the mass to gas percentage by the microorganisms loaded onto precipitated silica granules under these growth conditions as compared to the microorganisms under the control conditions. The increased amount of gas (˜1.6 times more) was being generated at hour 12, indicating an increased microbial activity with nutrients being converted to carbon dioxide and hydrogen. As this was a batch reactor and the food was in fixed supply, the amount of gas produced under the control conditions eventually caught up with the amount of gas produced under conditions using the DryLet® product. The rate of production of gases was accelerated by a factor of 1.5. During the time period between 8 to 14 hours, the relative rates of gas production were 0.075 g/hr and 0.05 g/hr by the microorganisms loaded onto precipitated silica granules and by the microorganisms under the control conditions respectively.

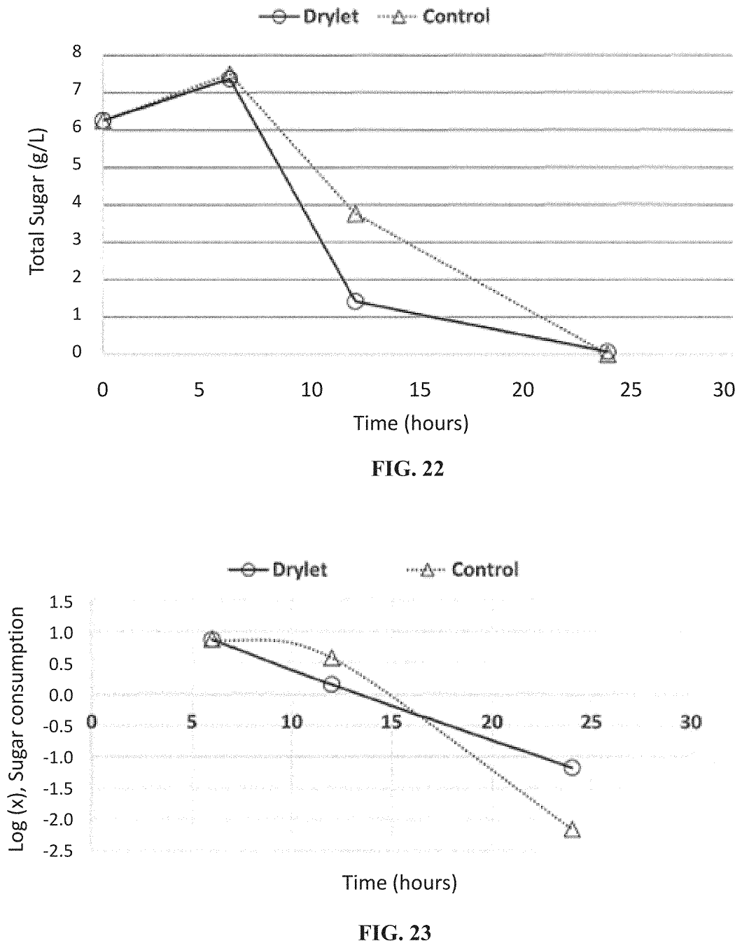

FIG. 22 is a graphical representation of the rate of sugar consumption under the two growth conditions. About 60% of the sugars were consumed by the microorganisms loaded onto precipitated silica granules in 12 hours while a similar amount, 60% of the sugars were consumed by the microorganisms in 18 hours growing under the control conditions.

FIG. 23 is a graphical representation of the logarithmic rate of sugar consumption under the two growth conditions. About 76.4% of the sugar was consumed by the microbes in the reactor containing the DryLet® product 12 hours, while only 37.4% of the sugar was consumed by the microbes in the control reactor at the same time. The rate of sugar consumption in the reactor with the DryLet® product was 0.382 g/l/hr and the rate of sugar consumption in the control reactor was 0.187 g/l/hr. Thus, there was 2.04 times more sugar consumption in the reactor containing the DryLet® product as compared to the control reactor.

### Example 2c

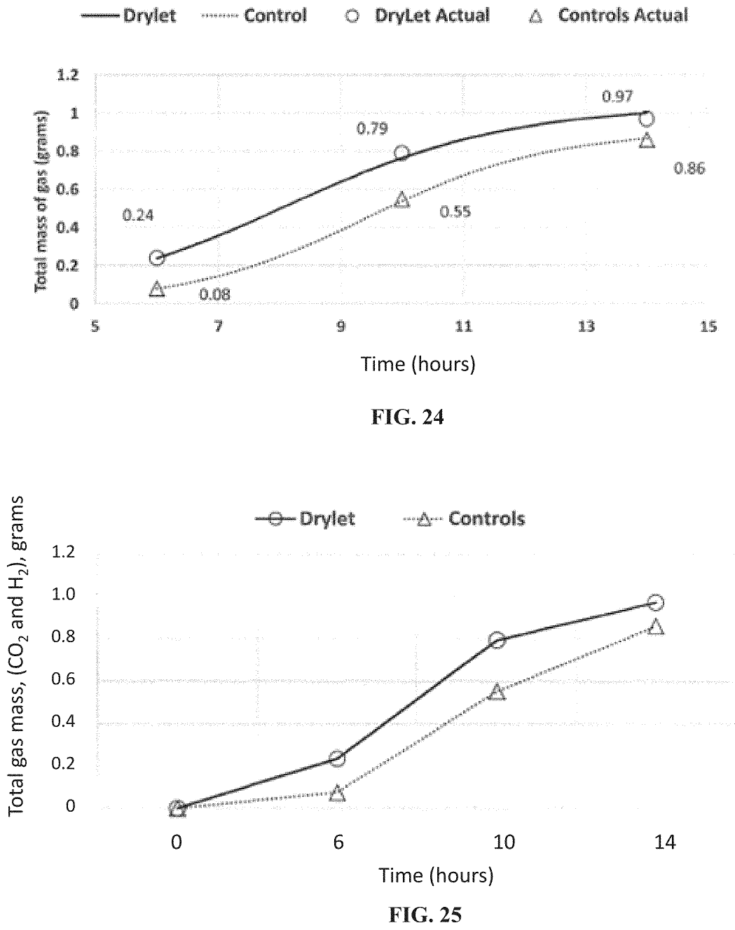

The growth kinetics of microorganisms growing under another set of two conditions were examined-first, microorganisms that have been introduced to the bioreactor as an inoculum from the mother culture; and second, same amount of microorganisms that have been loaded onto 6.5 grams of precipitated silica granules and then introduced to the bioreactor. FIG. 24 is a graphical representation of the growth kinetics of microorganisms growing under these two conditions. Compared to the previous sets of growth conditions, the energy solution consisted primarily of about six grams of glucose, sucrose, and fructose. The microorganisms loaded onto precipitated silica granules entered log phase in about five hours, while the microorganisms under the control conditions entered log phase about two hours later. The rate of gas production from hour 0 to hour 11 was at 0.16 g/hr and 0.11 g/hr for the reactors with DryLet® product and the control respectively. Thus, there was a 43% increase in the rate of gas production in the reactors with DryLet® product.

FIG. 25 is a graphical representation of the mass to gas percentage increase under the second growth conditions as compared to that under the control growth conditions. The same trend as seen in FIG. 27 was observed. The amount of gas produced at time zero under both conditions was practically zero. The rate of increase through the period 0 hours to 10 hours was about 0.08 g/hr and 0.055 g/hr when the microorganisms are loaded onto precipitated silica granules and the microorganisms under the control conditions respectively, thus, there was a 30% increase in the mass to gas percentage by the microorganisms loaded onto precipitated silica granules under these growth conditions as compared to the microorganisms under the control conditions.

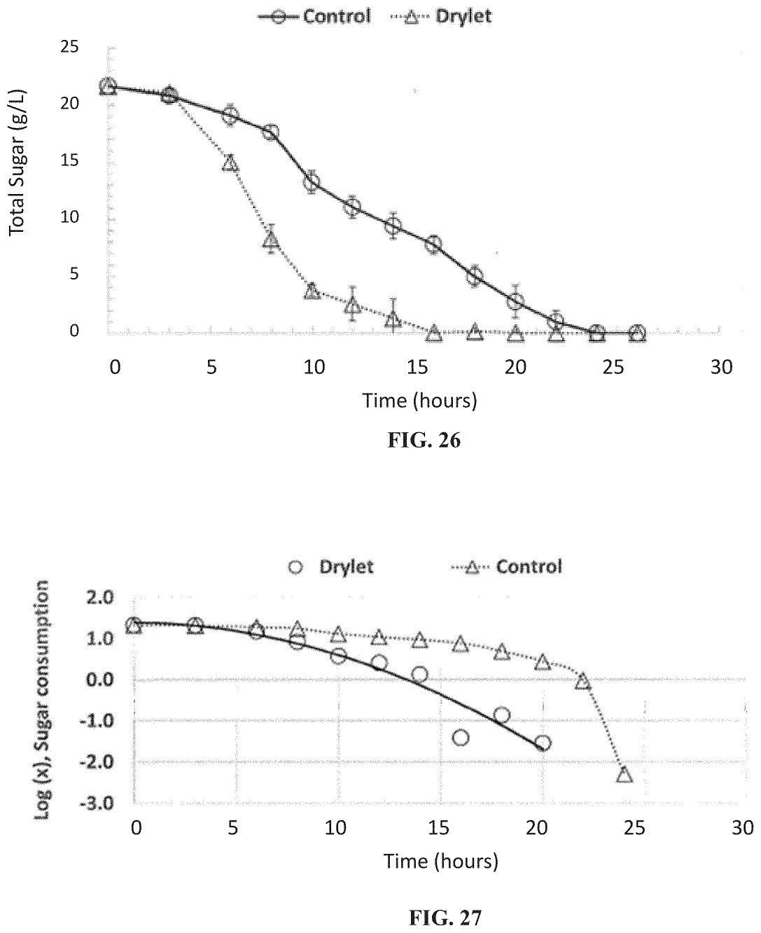

FIG. 26 is a graphical representation of the rate of sugar consumption under the two growth conditions. About 60% of the sugars were consumed by the microorganisms loaded onto precipitated silica granules in 7 hours under these growth conditions while a similar amount-60% of the sugars were consumed by the microorganisms in 15 hours growing under the control conditions. The rate of sugar consumption by the microorganisms loaded onto precipitated silica granules, which was an indication of the microbial population and carrying capacity, was shown to decrease at a rate of 2.5 g/l/hr until the sugar concentration was too low to sustain microbial growth. In same time period and through to 20 hours, the rate of sugar consumption by the microorganisms under the control conditions was 1.1 g/l/hr. There was a 2.3 times increase in rate of consumption of sugar by using DryLet® product.

FIG. 27 is a graphical representation of the logarithmic rate of sugar consumption under the two growth conditions.

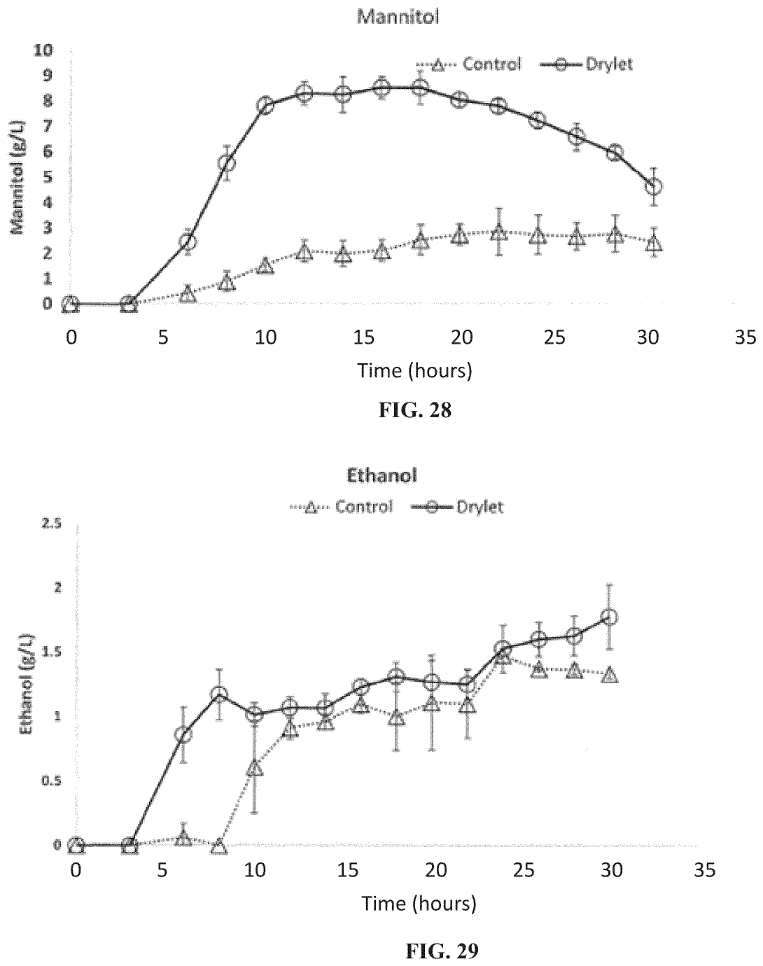

FIG. 28 is a graphical representation of the mannitol production under the two growth conditions. The initial rate of mannitol production by the microorganisms loaded onto precipitated silica granules under these growth conditions was 8 g/l/hr over the period of 3.5 to 11 hours, whereas the rate of mannitol production over the same time period under the control conditions was 0.28 g/l/hr. The concentration during the growth period reaches a much higher concentration relative to the control indicating a much higher carrying capacity when the microorganisms are loaded onto precipitated silica granules. The amount of mannitol production decreased with time after the peak because the feed sugar content was depleted.

FIG. 29 is a graphical representation of the ethanol production under the two growth conditions. Microorganisms loaded onto precipitated silica granules under these growth conditions showed a higher rate, faster time to ethanol production, along with an increase in carrying capacity for ethanol generation. The rate of production of ethanol was accelerated during the 3-hour to 8-hour time period, when the system reaches the stationary phase. The rate of ethanol production by microorganisms loaded onto precipitated silica granules was 0.3 g/l/hr, whereas the control has zero ethanol production in this period. Its production of ethanol was retarded by 4 hours under the control conditions and the growth rate through to 10 hours was 0.08 g/l/hr. The rate of ethanol production by microorganisms loaded onto precipitated silica granules was about 3.7 times faster than the control. As these are operated under batch reactor conditions with only limited amount of food, the rates reach a stationary phase and increasing concentration of the alcohol can slow the rate of production once a significant concentration has been reached. Thus, the carrying capacity of the system was increased when the microorganisms were loaded onto precipitated silica granules before being introduced to the bioreaction.

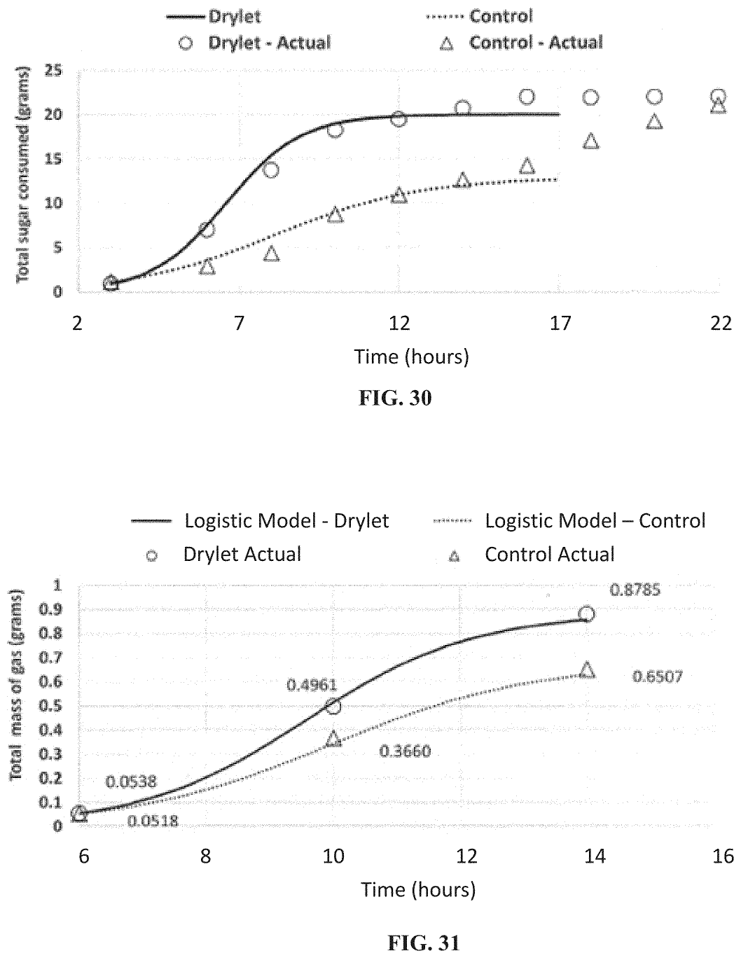

FIG. 30 is a graphical representation of the sugar uptake under the two growth conditions. The rate of sugar consumption by microorganisms loaded onto precipitated silica granules and by microorganisms under control conditions from time zero to 10 hours were approximately 2.4 g/l/hr and 1.1 g/l/hr, respectively. Thus, the carrying capacity of the system was increased at least by 2.2 fold when the microorganisms are loaded onto precipitated silica granules before being introduced to the bioreaction.

### Example 2d

The growth kinetics of microorganisms growing under two conditions were examined—first, microorganisms that have been introduced to the bioreactor as an inoculum from the mother culture; and second, same amount of microorganisms that have been loaded onto 6.5 grams of precipitated silica granules and then introduced to the bioreactor. FIG. 31 is a graphical representation of the growth kinetics of microorganisms growing under these two conditions. Compared to the previous sets of growth conditions, the energy solution here consisted primarily of about four grams of glucose, sucrose, maltodextrin, and P2 media. The microorganisms loaded onto precipitated silica granules entered log phase in about five hours, while the microorganisms under the control conditions entered log phase about two hours later.

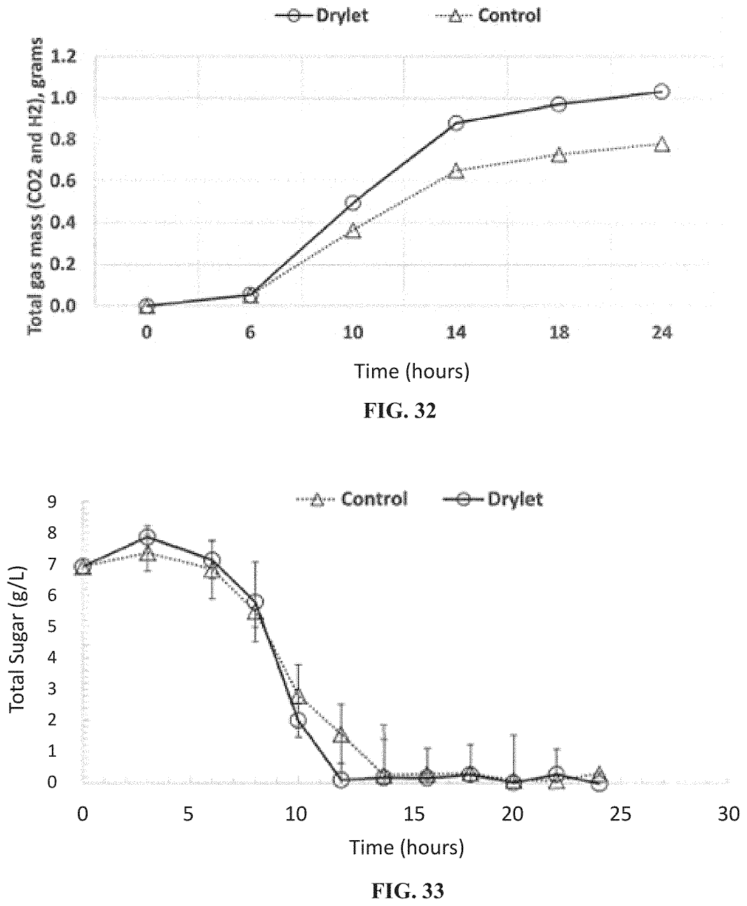

FIG. 32 is a graphical representation of the mass to gas percentage increase under the second growth conditions as compared to that under the control growth conditions. There was a 47% increase in the mass to gas percentage by the microorganisms loaded onto precipitated silica granules under these growth conditions as compared to the microorganisms under the control conditions.

FIG. 33 is a graphical representation of the rate of sugar consumption under the two growth conditions. About 60% of the sugars were consumed by the microorganisms loaded onto precipitated silica granules in 10 hours under these growth conditions while a similar amount (60%) of the sugars were consumed by the microorganisms in 11.5 hours growing under the control conditions.

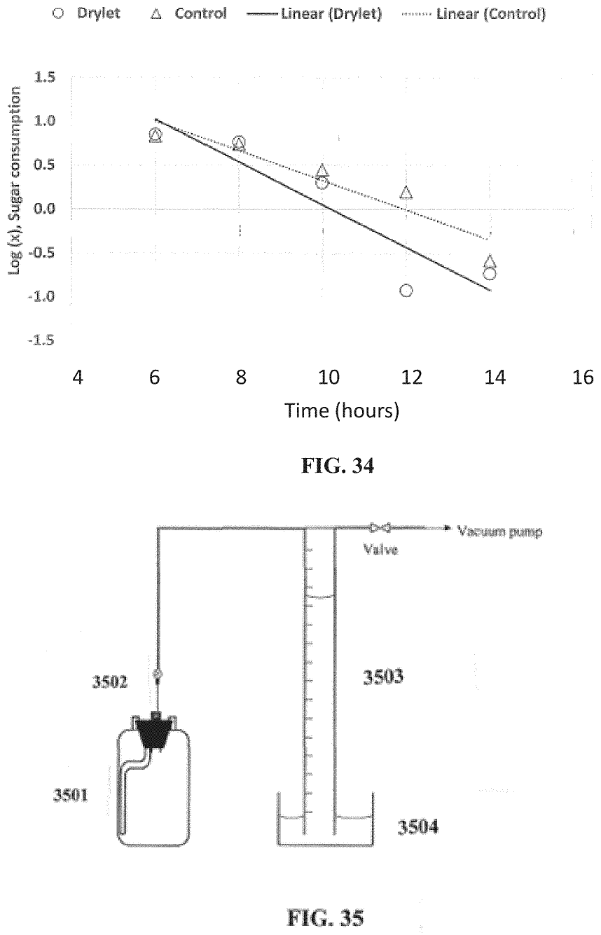

FIG. 34 is a graphical representation of the logarithmic rate of sugar uptake under the two growth conditions.

FIG. 35 is a diagrammatic representation of the experimental set-up designed to study the amount of gasses produced by the anaerobic systems. Each bioreactor 3501 was fitted with a tightfitting stopper and an exit tube 3502. The exit tube 3502 was connected to an inverted graduated cylinder 3503, placed in another vessel 3504 containing about 300 g/L of CaCb. In this instance, gas volumetric measurement was carried out by measuring the volume of liquid (cc) displaced by the gas produced with time along with the headspace in each reactor 3501. A 30 cc sample was removed for analysis by gas chromatography in the total amount of gas produced. Samples were analyzed according to previously described methods.

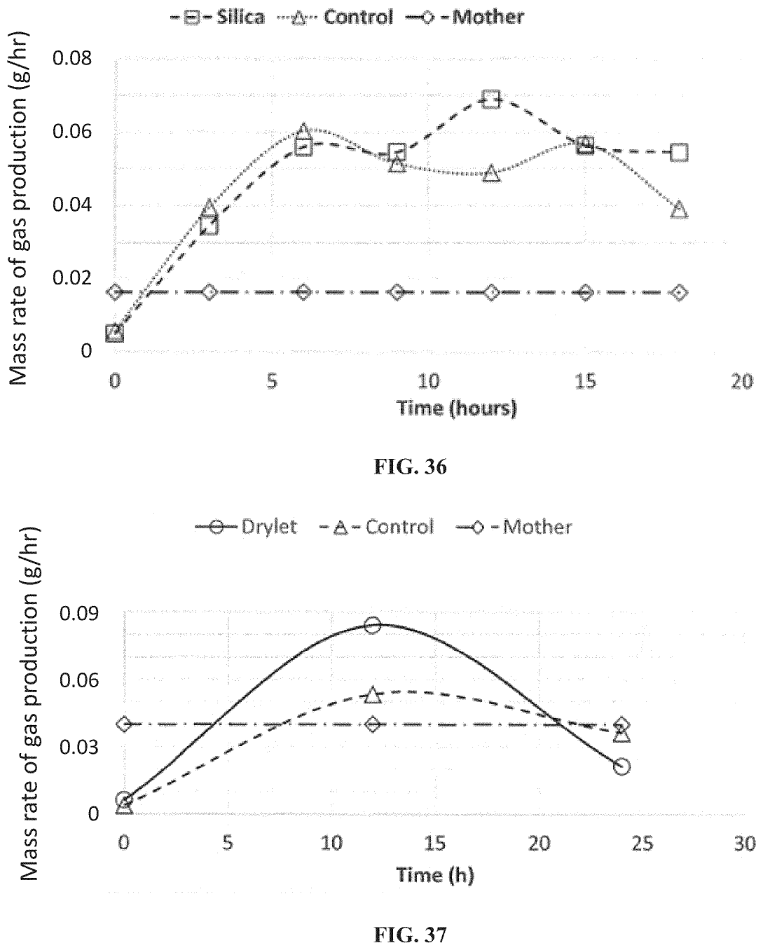

FIG. 36 is a graphical representation of the average mass rate of gasses produced under the two growth conditions described in Example 2a.

FIG. 37 is a graphical representation of the average mass rate of gasses produced under the two growth conditions described in Example 2b. The graphs show that a constant amount of gas was produced by the mother culture, while there was an increased rate of gas produced by the microorganisms that are loaded onto precipitated silica granules (DryLet® product) before being introduced to the bioreaction relative to the control samples.

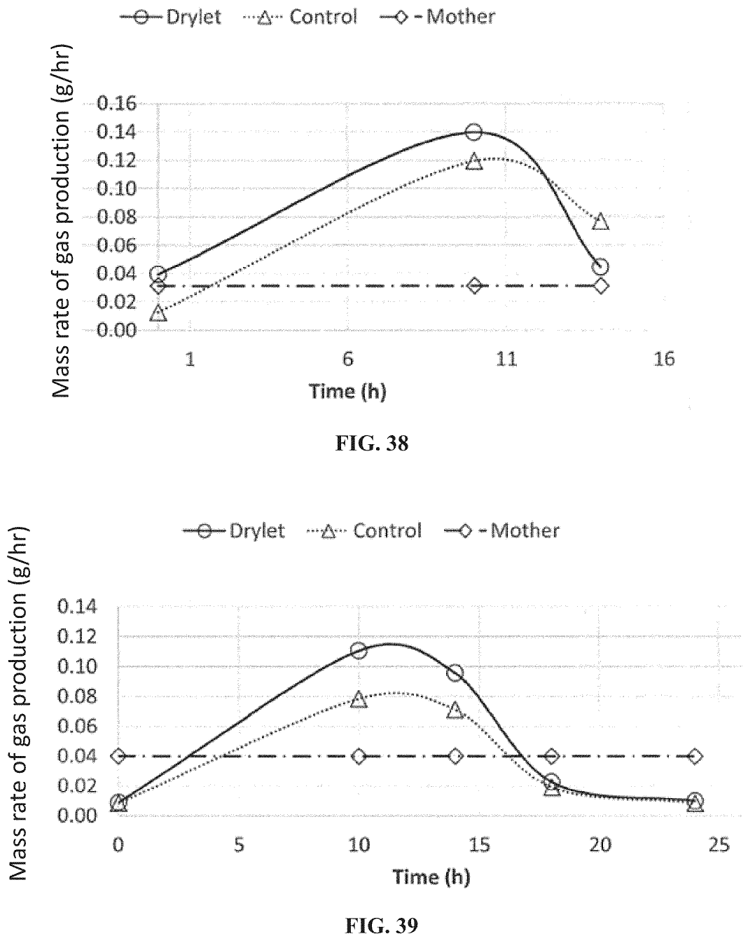

FIG. 38 is a graphical representation of the average mass rate of gasses produced under the two growth conditions described in Example 2c. The graphs show that a constant amount of gas was produced by the mother culture, while there was an increased rate of gas produced by the microorganisms that are loaded onto precipitated silica granules (DryLet® product) before being introduced to the bioreaction relative to the control samples.

FIG. 39 is a graphical representation of the average mass rate of gasses produced under the two growth conditions described in Example 2d. The graphs show that a constant amount of gas was produced by the mother culture, while there was an increased rate of gas produced by the microorganisms that are loaded onto precipitated silica granules (DryLet® product) before being introduced to the bioreaction relative to the control samples. The initial low value for gas evolution was because of the test dilutes the amount of culture initially. There was rapid gas increase followed by reaching a peak of gas evolution for both DryLet® product and control followed by decay as the microbes reach the stationary phase.

Summary of the results from Examples 2a-2d are presented in Table 25.

TABLE 25

| | | | Mass to | Mass to | | | | |
|---|---|---|---|---|---|---|---|---|
| | | | gas % | gas % | | | | |
| | | | increase | increase | Time | | Time to | |
| | | | over | over | to | Time to | reach | |
| | | | Mother | Controls | reach | consume | 60% | |
| | | | max | max | log | 60% of | Carrying | pH |
| | Mother | Food | (cc/hr) | (cc/hr) | phase | sugars | Capacity | range |
| Example 2a | GBS | Glucose, | 250% | 40% | 6 hrs | 9 hrs | 14.5 hrs | 4.25-7.5 |
| 3 Controls | | sucrose, | | | for | vs | for both | |
| and 3 | | fructose | | | both | 11.5 hrs | | |
| reactors with | | (~6 grams) | | | | | | |
| independent | | 2.4% | | | | | | |
| addition of | | wt./vol. | | | | | | |
| silica and | | | | | | | | |
| microbes | | | | | | | | |
| Example 2b | D1 | Sucrose, | 200% | 62% | 5 hrs | 12 hrs | 12 hrs | 5-7.5 |
| 3 Controls | | glucose, | | | vs | vs | vs | |
| and 3 | | maltodextrin, | | | 7 hrs | 18 hrs | 14.5 hrs | |
| reactors with | | P2 | | | | | | |
| DryLet ® | | media | | | | | | |
| product | | (~4 grams) | | | | | | |
| | | 1.6% | | | | | | |
| | | wt./vol. | | | | | | |
| Example 2c | D2 | Glucose, | 367% | 210% | 5 hrs | 7 hrs | 8.5 hrs | 5.75-7.5 |
| 3 Controls | | sucrose, | | | vs | vs | vs | |
| and 3 | | fructose | | | 7 hrs | 15 hrs | 10.5 hrs | |
| reactors with | | (~6 grams) | | | | | | |
| DryLet ® | | 2.4% | | | | | | |
| product | | wt./vol. | | | | | | |
| Example 2d | D1 | Sucrose, | 175% | 36% | 5 hrs | 10 hrs | 10 hrs | 5.25-7.5 |
| 3 Controls | | glucose, | | | vs | vs | vs | |
| and 3 | | maltodextrin, | | | 7 hrs | 11.5 hrs | 11.5 hrs | |
| reactors with | | P2 | | | | | | |
| DryLet ® | | media | | | | | | |
| product | | (~4 grams) | | | | | | |
| | | 2.4% | | | | | | |
| | | wt./vol. | | | | | | |

Other embodiments include a method of increasing the capacity for digestion in an anaerobic or anoxic process, as are commonly encountered in the wastewater industry in the form of anaerobic digesters and anoxic bioreactors (or anoxic “zones”). The microbial cultures used in Examples 2b, 2c, and 2d were all obtained directly from an anaerobic methane producing digester. The results show that digestion and gas production from such digesters can be greatly increased just as in the aerobic case in Example 1. CO₂ and H₂ gas formation are precursors to methane formation. By accelerating the rate and the amounts of CO₂ and H₂ in a bioreactor that is strictly anaerobic will lead to increased methane formation and more complete digestion of biomass contained in an anaerobic digester. Similarly, an anoxic bioreactor can also be accelerated by DryLet® product to accomplish denitrification at an accelerated catalyzed rate. All three conditions exist in wastewater lagoons, wherein the water near the surface may be well oxygenated, the solids blanket at the bottom of a lagoon will certainly be anaerobic (unless air is applied), and the water in between would be characterized as anoxic with regard to its dissolved oxygen levels.

### Example 3

Experiments were conducted to examine how growth patterns of a single microbial species can be altered by changing the amount of silica polymers, the mode of application of the microbes and the silica polymers, and the amount of nutrients in the system. For these experiments, the starting culture was Clostridium acetobutylicum ATCC 39236 initial prepared as instructed by ATCC. A batch of P2 media (Difco™ Reinforced Clostridial Medium) was inoculated from the mother culture. Over a period of −36 hours, samples were taken frequently (˜1.5 hours) and analyzed for the consumption of sugars and production of gaseous and liquid products. These data were analyzed to determine the growth rate of cells without DryLet® product (control) and with DryLet® product. The fermentation was performed in modified 1-L propylene centrifuge bottles sold by Beckman. These 1-L centrifuge bottles were placed horizontally in a Wheaton® modular cell culture roller bottle apparatus with multiple decks of parallel rollers that rotated the bottles at approximately 2.0 rpm. The incubator was a custom-made cabinet that contains the roller apparatus. The dimensions are similar to a home-refrigerator. The incubator circulates air through a bank of heaters to regulate the temperature to 40° C. Measurements of gas production were carried out as previously described above. Liquids were analyzed by gas chromatography. Acids were analyzed according to the methods employed by Earth Energy Renewables. The samples were filtered to remove debris. Phosphoric acid was added to acidify the solution so all acids are volatile. An internal standard was added for calibration purposes. A set of bioreactors were setup under conditions described in Table 26. As used in reactors described in Tables 26 and 27, the term “Low Silica” refers to reactor conditions, where 15 g of precipitated silica granules were added independently of the microbes to the reactor. The term “High Silica” refers to reactor conditions, where 30 g of precipitated silica granules were added independently of the microbes to the reactor. The term “Low DryLet® Product” refers to reactor conditions, where 15 g of precipitated silica granules were loaded with 15 mL of the microbial culture before addition to the reactor. The term “High DryLet® Product” refers to reactor conditions, where 30 g of precipitated silica granules were loaded with 30 mL of the microbial culture before addition to the reactor.

TABLE 26

| | Reactor No. | | | | | | | | | |
|---|---|---|---|---|---|---|---|---|---|---|
| | | | | | | | 4 | | | |
| | | | | | 3 | | Low Food | | | |
| | 1 | | 2 | | Low Food | | High | | 5 | |
| | Low Food | | Low Food | | Low Drylet ® | | Drylet ® | | Low Food | |
| Conditions | Low Silica | | High Silica | | Product | | Product | | Control | |
| Food | 15 | g/L | 15 | g/L | 15 | g/L | 15 | g/L | 15 | g/L |
| (concentration | | | | | | | | | | |
| of D-Mannose | | | | | | | | | | |
| in reactor) | | | | | | | | | | |
| Amount of | 30 | mL | 30 | mL | 15 | mL | 30 | mL | 30 | mL |
| Starter culture | | | | | | | | | | |
| Amount of | 15 | g | 30 | g | 15 | g | 30 | g | 0 | g |
| silica | | | | | | | | | | |

| | | | | | |
|---|---|---|---|---|---|
| Mode of | Microbes and | Microbes and | Microbes | Microbes | Only |
| delivery | precipitated | precipitated | loaded onto | loaded onto | Microbes |
| | silica granules | silica granules | precipitated | precipitated | |
| | added | added | silica granules | silica granules | |
| | independently | independently | before | before | |
| | to reactor | to reactor | addition to | addition to | |
| | | | reactor | reactor | |

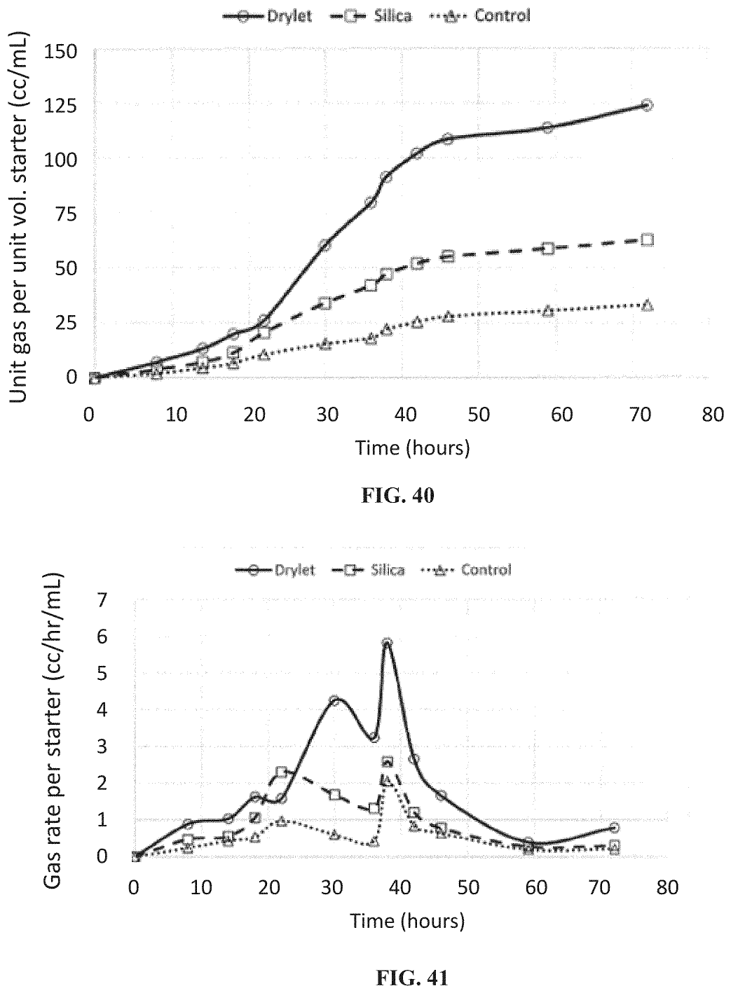

The metabolic kinetics of microorganisms growing under three conditions (Reactors 1, 3, and 5 in Table 26) were examined. FIG. 40 is a graphical representation of the total gas produced per unit vol. of starting culture when there was lower amount food in the system (15 g/l). About 15 mL of the starter culture was loaded onto silica granules (DryLet® product) before supplying them to Reactor 3, whereas 30 mL was loaded along with the 15 g of silica into Reactor 1. Using the DryLet® product led to almost 200% more gas per unit volume of starting culture in Reactor 3 as compared to the amount of gas produced in Reactor 1 and 5. FIG. 41 is a graphical representation of the rate of gas (cc/hr) generated under three conditions (Reactors 1, 3, and 5) normalized to the amount of unit volume in the starting culture as measured by milliliters (ml). Using the DryLet® product led to a gas production rate that was 2.24 times greater than the gas production rate in Reactor 1 and 5.

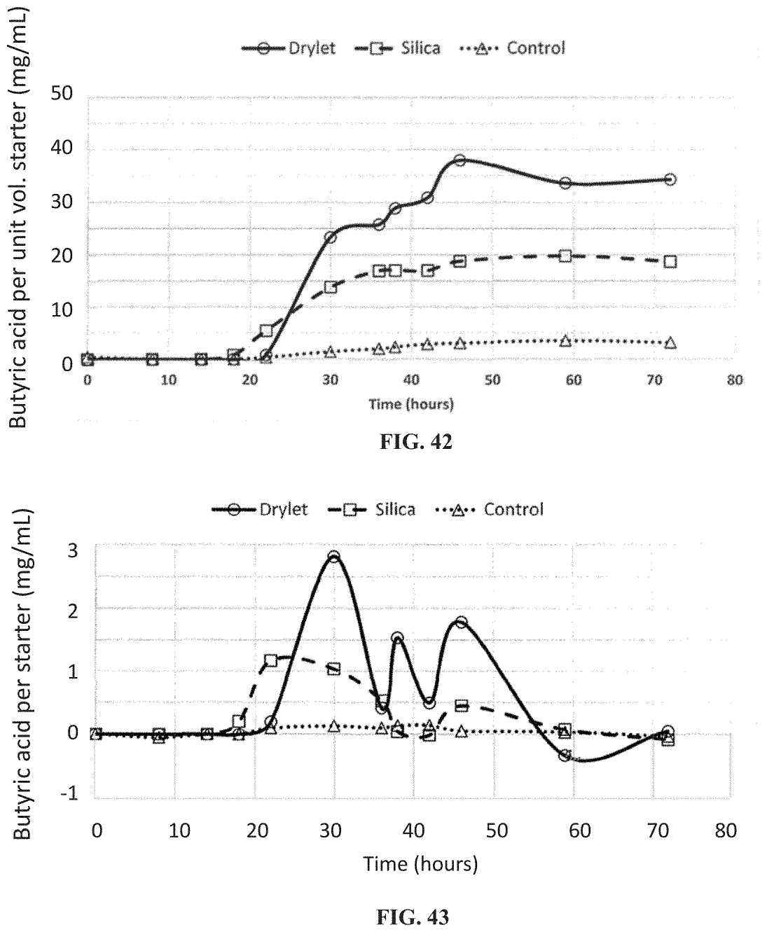

FIG. 42 is a graphical representation of butyric acid production per unit volume of starting culture when there was lower amount food in the system (15 g/l). Using the DryLet® product generated 2.02 times more butyric acid (mg/ml) than the acid produced in Reactor 1 from time 22 hrs until reaction completion at time 72 hours. FIG. 43 is a graphical representation of the rate of butyric acid production per unit volume of starting culture when there was lower amount food in the system (15 g/l). Using the Dry Let® product, the rate of butyric acid production was 2.41 times greater than the acid production rate in Reactor 1 from time 22 hrs until reaction completion at time 72 hours.

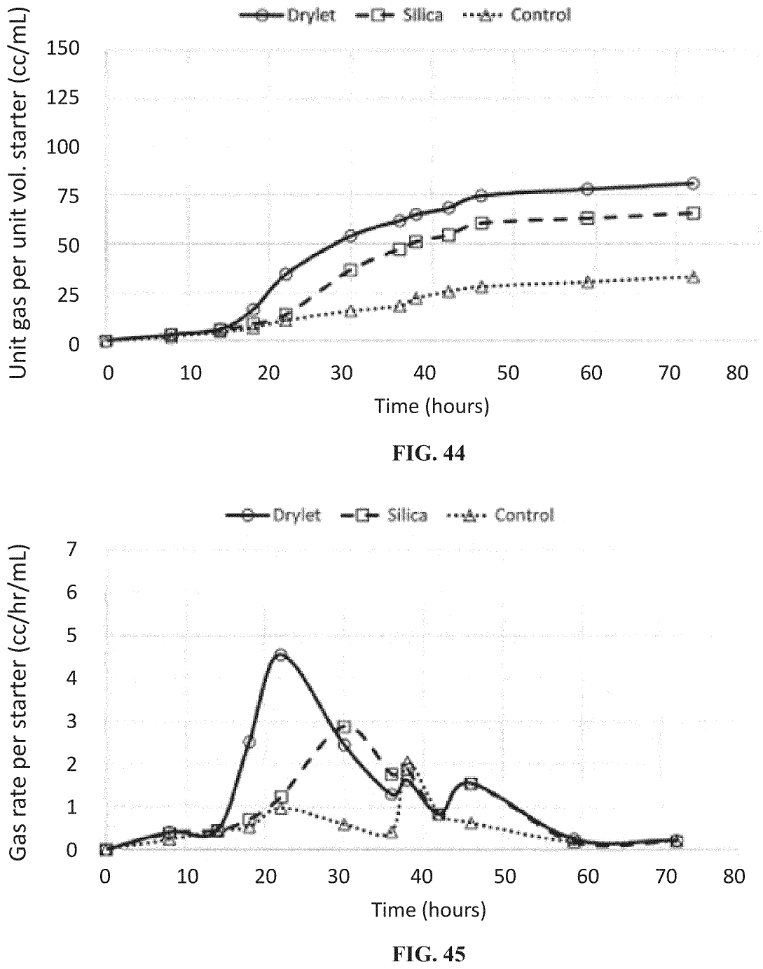

The metabolic kinetics of microorganisms growing under three conditions (Reactors 2, 4, and 5 in Table 26) were examined. FIG. 44 is a graphical representation of the total gas produced per unit vol. of starting culture when there was lower amount food in the system (15 g/1) but the amount of silica present was increased to 30 g. Using the DryLet® product led to almost 46% more gas per unit volume of starting culture in Reactor 4 as compared to the amount of gas produced in Reactor 2 and 5. Even during stationary phase, using the DryLet® product produced a steady rate of 20% to 30% more gas as compared to the amount of gas produced in Reactor 2 until the food was exhausted. FIG. 45 is a graphical representation of the rate of gas (cc/hr) generated under three conditions (Reactors 2, 4, and 5 in Table 26) normalized to the amount of unit volume in the starting culture as measured by milliliters (ml). Using the DryLet® product led to a gas production rate that was 1.58 times greater than the gas production rate in Reactor 2 and 5. There was also an earlier peak in production in Reactor 4. Using the DryLet® product led to a gas production rate of 4.56 cc/hr/ml at approximately 22 hrs, as compared to a gas production rate of 2.88 cc/hr/ml at approximately 30 hrs. This shows that the metabolic process for conversion of the food started about 8 hours earlier in Reactor 4 than in Reactor 2 and the peak gas rate was 58.3% greater in Reactor 4.

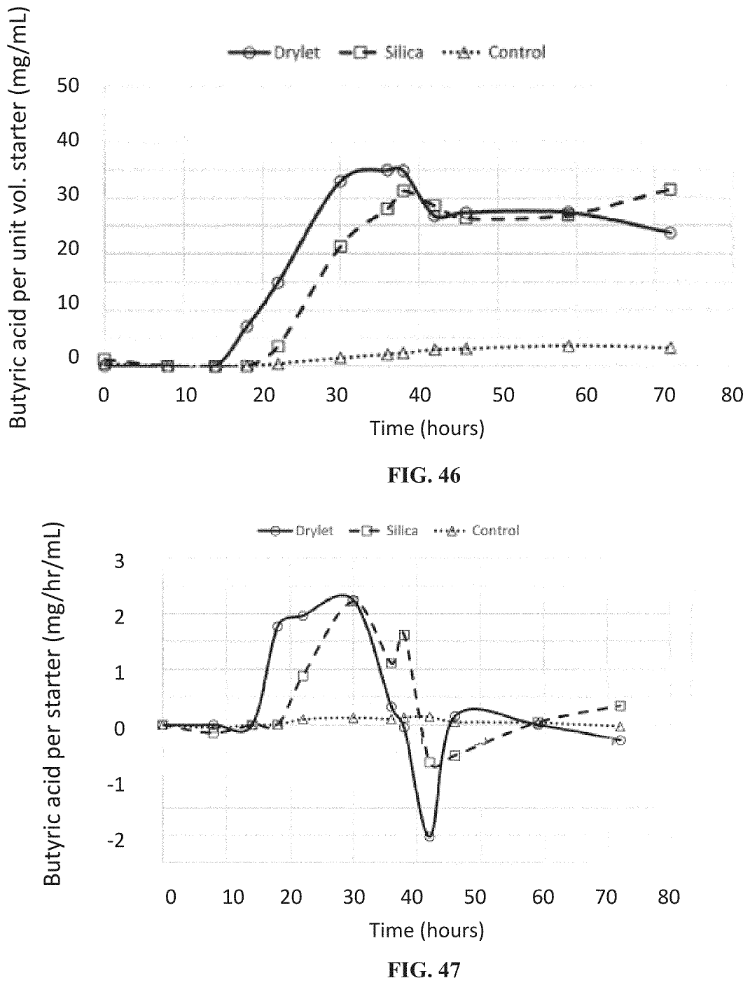

FIG. 46 is a graphical representation of butyric acid production per unit volume of starting culture when there was lower amount food in the system (15 g/l) but the amount of silica present was increased to 30 g. In Reactor 4, butyric acid production started 4 hours earlier (time=14 hours) as compared to the butyric acid production in Reactor 2. And, there was higher butyric acid production per unit volume of starting culture (mg/ml)-34.92 mg/ml in Reactor 4 as compared to the 31.27 mg/ml in Reactor 2; an 11.7% increase in butyric acid production.

FIG. 47 is a graphical representation of the rate of butyric acid production per unit volume of starting culture when there was lower amount food in the system (15 g/l) but the amount of silica present was increased to 30 g. Using the DryLet® product, the rate of butyric acid production was about 1.77 to 2.23 mg/hr/ml and was sustained for 12 hours in Reactor 5. Although the use of microbes and silica independently in Reactor 2 also led to a similar peak rate of butyric acid production of 2.22 mg/hr/ml, this rate was reached about four hours later and lasted only about an hour.

A set of bioreactors were setup under conditions described in Table 27.

TABLE 27

| | Reactor No. | | | | | | | | | |
|---|---|---|---|---|---|---|---|---|---|---|
| | | | | | | | 9 | | | |
| | | | | | 8 | | High Food | | | |
| | 6 | | 7 | | High Food | | High | | 10 | |
| | High Food | | High Food | | Low Drylet ® | | Drylet ® | | High Food | |
| Conditions | Low Silica | | High Silica | | Product | | Product | | Control | |
| Food | 30 | g/L | 30 | g/L | 30 | g/L | 30 | g/L | 30 | g/L |
| (concentration | | | | | | | | | | |
| of D-Mannose | | | | | | | | | | |
| in reactor) | | | | | | | | | | |
| Amount of | 30 | mL | 30 | mL | 15 | mL | 30 | mL | 30 | mL |
| Starter culture | | | | | | | | | | |
| Amount of | 15 | g | 30 | g | 15 | g | 30 | g | 0 | g |
| silica | | | | | | | | | | |

| | | | | | |
|---|---|---|---|---|---|
| Mode of | Microbes and | Microbes and | Microbes | Microbes | Only |
| delivery | precipitated | precipitated | loaded onto | loaded onto | Microbes |
| | silica granules | silica granules | precipitated | precipitated | |
| | added | added | silica granules | silica granules | |
| | independently | independently | before | before | |
| | to reactor | to reactor | addition to | addition to | |
| | | | reactor | reactor | |

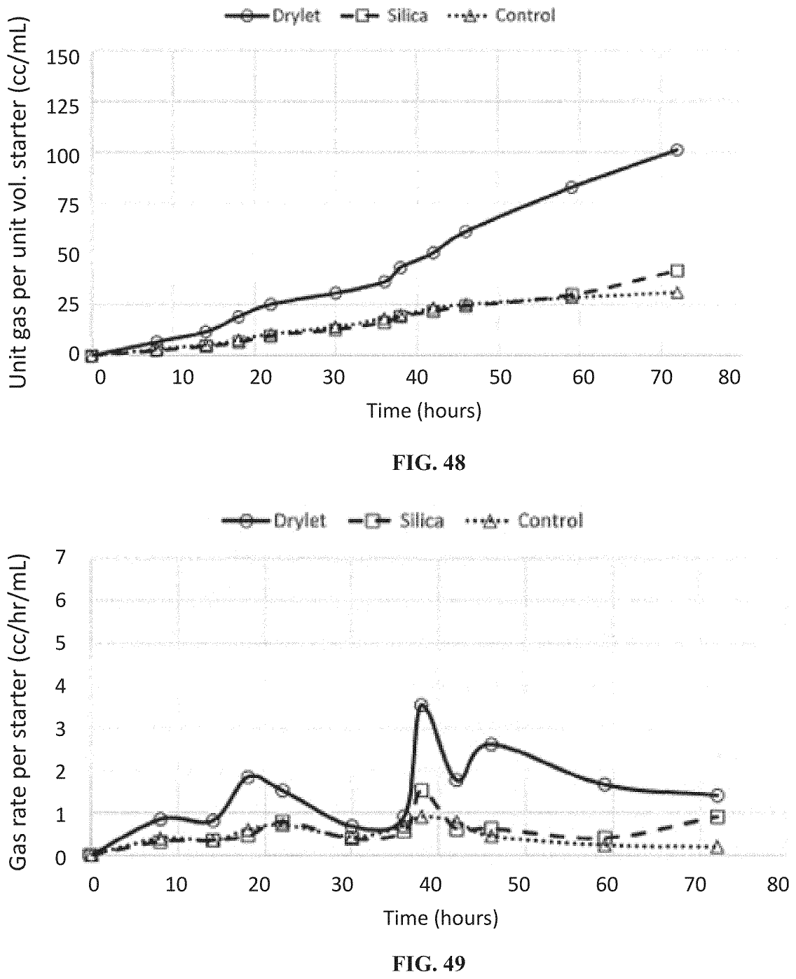

The metabolic kinetics of microorganisms growing under three conditions (Reactors 6, 8, and 10 in Table 27) were examined. FIG. 48 is a graphical representation of the total gas produced per unit vol. of starting culture when there was larger amount food in the system (30 g/l). About 15 mL of the starter culture was loaded onto silica granules (DryLet® product) before supplying them to Reactor 8, whereas 30 mL was loaded along with the 15 g of silica into Reactor 6. From initial gas production at time=8 hours for all reactors, more gas (cc) per unit volume of starting culture (ml) was produced in Reactor 8 as compared to Reactors 6 and 10. Using the DryLet® product led to almost 117% more gas per unit volume of starting culture in Reactor 8 as compared to the amount of gas produced in Reactor 6 and 10.

FIG. 49 is a graphical representation of the rate of gas (cc/hr) generated under three conditions (Reactors 6, 8, and 10 in Table 27) normalized to the amount of unit volume in the starting culture as measured by milliliters (ml). Using the DryLet® product led to a gas production rate that was 2.33 times greater than the gas production rate in Reactor 6. In Reactor 8, the peak gas production rate was 3.55 cc/hr/ml (at time=38 hrs), whereas in Reactor 6, the peak gas production rate was 1.52 cc/hr/ml (time=38 hrs). Moreover, the metabolic process for significant conversion of the food started earlier in Reactor 8 at t=18 hours than in Reactor 6 at t=38 hours. Reactor 8 had a maximum rate of gas production that was almost 2.34 times greater than the gas production rate in Reactor 6.

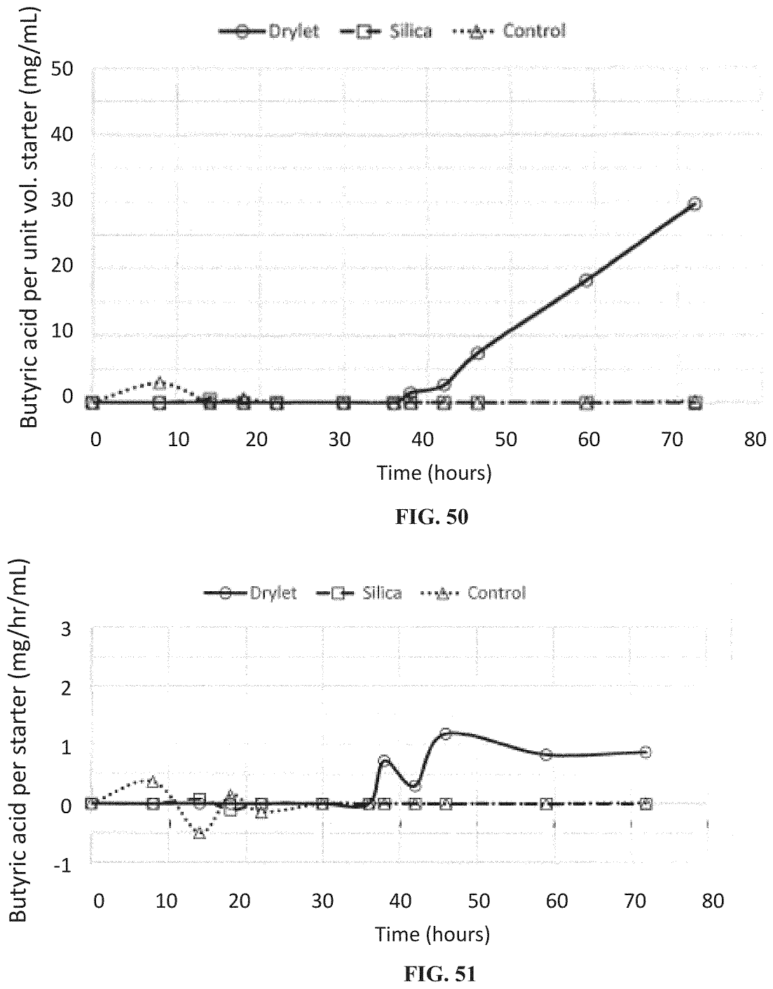

FIG. 50 is a graphical representation of butyric acid production per unit volume of starting culture when there was larger amount food in the system (30 g/l). Butyric acid production was negligible in Reactors 6 and 10. Only Reactor 8 produced butyric acid of about 29.66 mg/ml. FIG. 51 is a graphical representation of the rate of butyric acid production per unit volume of starting culture. The maximum rate was 1.19 mg/hr/ml in Reactor 8, while the rates were negligible in Reactors 6 and 10 due to lack of butyric acid production.

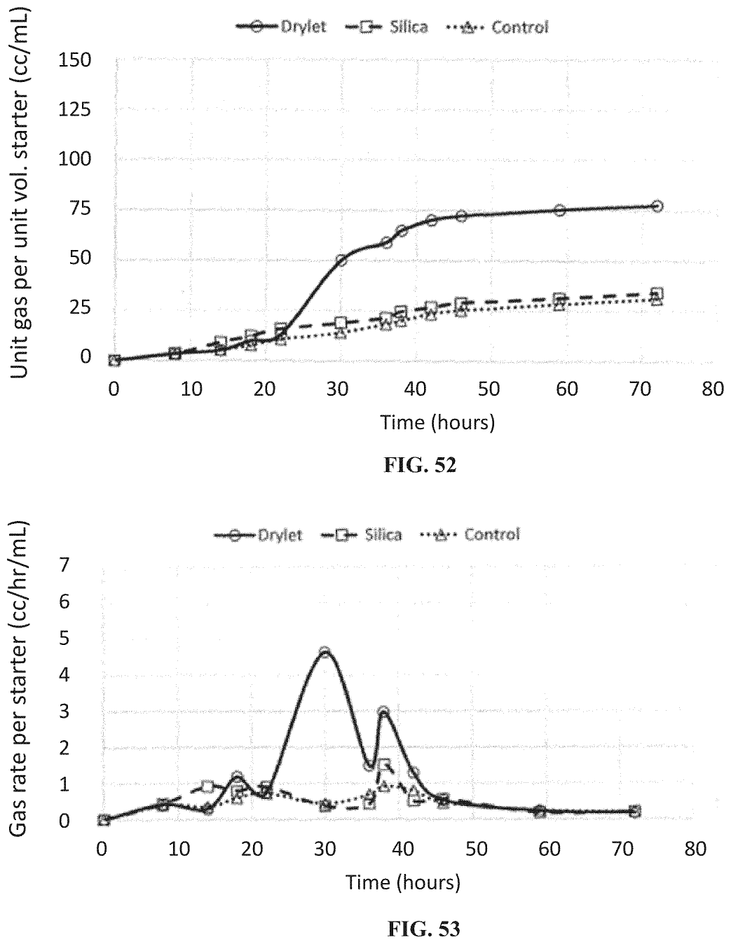

The metabolic kinetics of microorganisms growing under three conditions (Reactors 7, 9, and 10) were examined. FIG. 52 is a graphical representation of the total gas produced per unit vol. of starting culture when there was larger amount food in the system (30 g/l) and the amount of silica present was increased to 30 g. Use of the DryLet® product led to almost 144% more gas per unit volume of starting culture in Reactor 9 as compared to the amount of gas produced in Reactors 7 and 10. Use of the DryLet® product exhibited a significant increase in total gas produced per unit volume of starting culture (ml) at time=30 hours and was maintained until completion at time=72 hours.

FIG. 53 is a graphical representation of the rate of gas (cc/hr) generated under three conditions (Reactors 7, 9, and 10 in Table 27) normalized to the amount of unit volume in the starting culture as measured by milliliters (ml). Using the DryLet® product led to a gas production rate that was 3.05 times greater than the gas production rate in Reactor 7 and 10. There was also an earlier peak in production in Reactor 9. Using the DryLet® product led to a gas production rate of 4.64 cc/hr/ml at approximately 30 hrs, as compared to a gas production rate of 1.52 cc/hr/ml at approximately 38 hrs. This shows that the metabolic process for conversion of the food started about 8 hours earlier in Reactor 9.

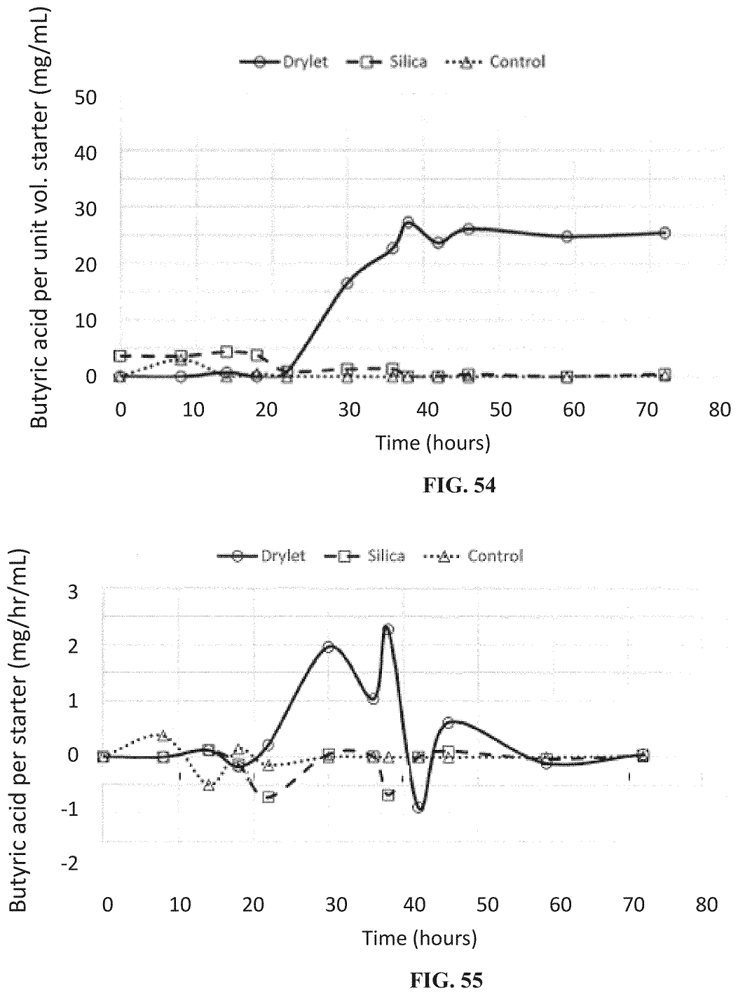

FIG. 54 is a graphical representation of butyric acid production per unit volume of starting culture when there was larger amount food in the system (30 g/l) and the amount of silica present was increased to 30 g. Butyric acid production was negligible in Reactors 7 and 10. Only Reactor 9 produced butyric acid at time=36 hours to 72 hours in a range of 22.7 to 27.22 mg/ml. FIG. 55 is a graphical representation of the rate of butyric acid production per unit volume of starting culture. The maximum rate was 1.95-2.27 mg/hr/ml in Reactor 9, while the rates were negligible in Reactors 7 and 10 due to lack of butyric acid production.

This Example demonstrates that the addition of silica polymers to a bioreactor containing microbes increases microbial growth and several metabolic processes as compared to a control bioreactor without the silica polymers. The use of DryLet® product dramatically increased microbial growth and the rate of production of products by the microbes as compared to the control bioreactors.

As used herein, “about” refers to a degree of deviation based on experimental error typical for the particular property identified. The latitude provided the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” shall expressly include “exactly,” consistent with the discussion below regarding ranges and numerical data.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3.5, and 4 and sub-ranges such as from 1-3, from 2-4, and from 3-5, etc. Additionally, a numerical range with a lower end of “0” can include a sub-range using “0.1” as the lower end point.

While the disclosure has been described with reference to certain examples, those skilled in the art will appreciate that various modifications, changes, omissions, and substitutions can be made without departing from the spirit of the disclosure. It is intended, therefore, that the present disclosure be limited only by the scope of the following claims.

1. (canceled)

2. A system for increasing a carrying capacity of a wastewater treatment plant, the system comprising:
a bioreactor comprising silica polymers containing microorganisms and wastewater, wherein the bioreactor is configured to produce a suspension with at least two times more total suspended solids upon reaching steady state than a control bioreactor containing microorganisms without the silica polymers; and
a first solid-liquid separator configured to receive the suspension from the bioreactor and produce a first fraction containing suspended solids and a treated water stream.

3. The system of claim 2, further comprising an aeration basin configured to mix the wastewater and the silica polymers containing microorganisms.

4. The system of claim 2, wherein the silica polymers are precipitated silica granules having a porous structure and loaded with microorganisms throughout the pores of the precipitated silica granules.

5. The system of claim 2, wherein a portion of the fraction containing suspended solids is recycled to the bioreactor.

6. The system of claim 5, further comprising an additional bioreactor configured to receive another portion of the fraction containing suspended solids and produce digested products under anaerobic or anoxic conditions.

7. The system of claim 6, further comprising a second solid-liquid separator configured to receive the digested product and produce a water stream and a filter cake.

8. The system of claim 2, wherein the bioreactor consumes at least about 20% less of flocculating agent than the control bioreactor.

9. The system of claim 2, wherein the silica polymers are loaded with nutrients.

10. The system of claim 2, wherein the carrying capacity of the bioreactor is at least 1.5 times greater than the carrying capacity of the control bioreactor.

11. The system of claim 2, wherein the microorganisms are a consortium of native, non-pathogenic microbial species.

12. The system of claim 2, wherein the microorganisms are bacteria.

13. The system of claim 2, wherein the bioreactor produces at least about 25% less sludge than the control bioreactor.

14. The system of claim 2, wherein the bioreactor is an aerobic bioreactor.

15. The system of claim 2, wherein the bioreactor is an anaerobic bioreactor.

16. The system of claim 2, wherein the bioreactor comprises aerobic, anaerobic, and anoxic conditions.

17. The system of claim 2, wherein the bioreactor is a wastewater lagoon.

18. The system of claim 2, the bioreactor is configured to operate in a batch mode.

19. The system of claim 2, the bioreactor is configured to operate in a continuous mode.
